(19) United States
(12) Patent Application Publication
(10) Pub. No.: US 2026/0234201 A1
(43) Pub. Date: Aug. 13, 2026

(54) DARPINS FOR USE IN REDUCING RENAL ACCUMULATION OF DRUGS

(71) Applicant: MOLECULAR PARTNERS AG, Zürich-Schlieren (CH)

(72) Inventors: Andreas BOSSHART, Zürich-Schlieren (CH); Daniel STEINER, Zürich-Schlieren (CH)

(21) Appl. No.: 19/156,043

(22) PCT Filed: Feb. 26, 2024
(86) PCT No.: PCT/EP2024/054797

§ 371 Date: Aug. 13, 2025

(30) Foreign Application Priority Data
Feb. 27, 2023 (EP) 23158902.9

(51) Int. Cl. C07K 14/47; A61K 38/00; A61P 43/00

(52) U.S. Cl. CPC C07K 14/4702; A61P 43/00; A61K 38/00

(57) ABSTRACT

The present invention relates to designed ankyrin repeat domains and designed ankyrin repeat proteins for use in reducing accumulation of a therapeutic and/or diagnostic agent in the kidney of a subject treated with said agent. Preferably such therapeutic and/or diagnostic agent comprises a drug moiety such as a radionuclide or a cytotoxin. In addition, the invention provides recombinant proteins comprising such repeat domains, nucleic acids encoding such repeat domains or recombinant proteins, recombinant expression vectors, host cells, and pharmaceutical compositions comprising such repeat domains, recombinant proteins, nucleic acids or recombinant expression vectors, as well as the use of such repeat domains, recombinant proteins or pharmaceutical compositions in methods for reducing accumulation of a therapeutic and/or diagnostic agent in the kidney of a subject treated with said agent.

### CROSS-REFERENCE TO RELATED APPLICATION

The present application claims the benefit of and priority from European patent application EP23158902.9 filed on 27 Feb. 2023 with the European Patent Office. The content of European patent application EP23158902.9 is incorporated herein by reference in its entirety, including all tables, figures, and claims.

### FIELD OF THE DISCLOSURE

The present invention relates to designed ankyrin repeat domains and designed ankyrin repeat proteins for use in reducing accumulation of a therapeutic and/or diagnostic agent in the kidney of a subject treated with said agent. Preferably such therapeutic and/or diagnostic agent comprises a drug moiety such as a radionuclide or a cytotoxin. In addition, the invention provides recombinant proteins comprising such repeat domains, nucleic acids encoding such repeat domains or recombinant proteins, recombinant expression vectors, host cells, and pharmaceutical compositions comprising such repeat domains, recombinant proteins, nucleic acids or recombinant expression vectors, as well as the use of such repeat domains, recombinant proteins or pharmaceutical compositions in methods for reducing accumulation of a therapeutic and/or diagnostic agent in the kidney of a subject treated with said agent.

### BACKGROUND

Targeted radiopharmaceuticals have emerged as a promising tool in the diagnosis and treatment of cancers. Such radiopharmaceuticals typically consist of a radioactive molecule (e.g. radionuclide) linked to a binding molecule (e.g. antibodies or fragments thereof, protein scaffolds, peptides or small molecules). By combining a specificity for a biological target with an ionizing radiation source, radiopharmaceuticals can concentrate radiation emission in the vicinity of a biomarker of interest. High target selectivity and tumor retention, low uptake in non-tumoral organs and tissues and a fast clearance are desired characteristics of precise cancer diagnostics (e.g. the radioactive agent reveals the tumor location) and/or therapy (e.g. the radioactive agent damages the tumor) with radiopharmaceuticals.

Early work in radioimmunotherapy highlighted important drawbacks of using radiolabeled antibodies, such as a slow extravasation and slow clearance of intact antibodies from the blood due to their size (150 kDa). With antibody based-radiopharmaceuticals, an optimal tumor-to-background ratio can typically be reached only after several days, therefore inducing indirect damage to radiosensitive organs and tissues such as bone marrow. Subsequently, alternative binders of lower molecular weights were developed to improve pharmacokinetics and increase tumor-to-normal tissue dose ratios. Drug clearance in the context of radiolabeled peptides, including small antibody fragments, is predominantly driven by renal excretion which involves the physiological processes of glomerular filtration, active tubular secretion, and tubular reabsorption. Glomerular filtration ensures that circulating cells and valuable macromolecular components of blood plasma are selectively retained based on molecular size. Molecules weighing more than 70 kDa or being larger than 4.2 nm in radius, and those bound to plasma proteins (such as albumin) undergo negligible glomerular filtration (Parihar, A. S. et al., Translational Oncology 15.1 (2022): 101295).

Several radiopharmaceuticals, due to their inherent properties, are retained within the kidneys, herewith contributing to an increased radiation absorbed dose to the kidneys. Even though small format binding molecules with a lower molecular weight can provide a combined advantage of rapid targeting and rapid clearance with minimal uptake in normal tissues or organs early after injection, their use also induces undesired high renal accumulation of radioactivity thereby hindering their broader clinical application. Both the choice of radionuclide and the nature of the binding molecule may impact the severity of nephrotoxicity resulting from such high radioactivity accumulation in kidneys (Chigoho, D. M. et al., Current opinion in chemical biology 63 (2021): 219-228).

A key contributor to nephrotoxicity is the process of renal reabsorption. Low to moderate molecular weight radiolabeled molecules are readily filtered through the glomerulus and are subsequently reabsorbed by proximal tubular cells and subsequently catabolized in the cells. After proteolytic degradation in lysosomes, radiolabeled catabolites are released and, depending on their physical properties, are either freely washed out of the cells (non-residualizing radionuclide) or are retained intracellularly (residualizing radionuclide). Residualizing radionuclides are typically advantageous from the viewpoint of tumor cytotoxicity but can increase the toxicity profile due to off-target localization in normal tissues.

To palliate such renal retention, pharmacological and/or physicochemical approaches have been tried, for instance by acetylation of ^{99m}Tc-labeled humanized anti-Tac monoclonal antibody Fab, and further by co-injection of lysine (Kim, M. K, et al., Nuclear medicine and biology, 29.2 (2002): 139-146). Moreover, the effect of single amino acid substitutions on renal radioactivity accumulation has been studied by Akizawa et al., in the context of a very short, 8 amino acid peptide (¹¹¹In-DTPA-conjugated octreotide derivatives) (Akizawa, H., et al., Nuclear medicine and biology 28.7 (2001): 761-768). Further, co-administration of Gelofusine (succinylated gelatine), which is suspected of interfering with the megalin/cubulin-mediated reabsorption process, has been tried (Geenen, L. et al., Nuclear Medicine and Biology 102 (2021): 1-11). However, administration of such mitigation compounds acting on various parts of the reabsorption system in the kidney was shown not to be effective for all radiopharmaceuticals.

A study on the prevention of renal uptake of ^{99m}Tc-labeled designed ankyrin repeat proteins (DARPins) showed that common clinical strategies were not effective for reducing kidney uptake of these radiolabeled DARPins in mice. More specifically, co-injection of lysine or Gelofusine did not reduce renal uptake. Pre-administration of high doses of maleate or fructose, which inhibit ATP-mediated endocytosis, resulted in a reduction of kidney uptake of these protein scaffolds, but at a required dose not suitable for a clinical application. No other compounds were effective. As such, according to the authors, the study suggested that the renal uptake of ^{99m}Tc-labeled DARPins proceeds through a mechanism independent of DARPin structure and binding site composition. (Altai, M. et al., EJNMMI research 10.1 (2020): 1-8). More recently, a reduced uptake of a radiolabeled DARPin was observed in mice, but the relevance of this observation for therapeutic and/or diagnostic approaches remained unclear (Fay et al., Molecular Pharmaceutics (2022)).

Thus, despite attempts to reduce renal accumulation and mitigate nephrotoxicity with pharmacological and/or physicochemical approaches, this could not consistently be achieved and nephrotoxicity remains a hurdle for the application of radiopharmaceuticals in radiopharmaceutical therapy or diagnostics. Similar considerations apply to pharmaceuticals in which a small format target-specific binder is linked to a non-radioactive drug moiety, such as, e.g., a cytotoxic molecule, instead of a radionuclide (Richards, D. A. Drug Discovery Today: Technologies 30 (2018): 35-46).

Taken together, there remains a need for new approaches for reducing kidney accumulation of toxic drug moieties comprised in therapeutic or diagnostic agents administered to subjects. Examples of such toxic drug moieties are radionuclides or cytotoxic molecules. Such approaches may be useful in therapeutic or diagnostic applications, such as, for example, applications involving radiopharmaceuticals or cytotoxic drug-conjugates.

### SUMMARY

The present invention relates to the use of designed ankyrin repeat domains or proteins to reduce accumulation of a therapeutic and/or diagnostic agent (or of a drug moiety comprised in such agent) in the kidney of a subject treated with said agent. Examples of such agents include radiotherapeutic agents, radiodiagnostic agents or cytotoxic drug-conjugates. The designed ankyrin repeat domains and proteins described herein can be used to reduce renal accumulation of agents comprising a drug moiety (or of the drug moiety itself). Such a agent may be a radiolabeled or cytotoxin linked designed ankyrin repeat protein (DARPin). The inventors unexpectedly found that renal uptake of a radiolabeled agent can be significantly reduced by co-administration of a non-radiolabeled (cold) DARPin. This reductive effect was observed independently of the cold DARPin's binding characteristics and was in particular observed when the cold DARPin had no defined binding specificity. Without being bound by theory, the methods and uses of designed ankyrin repeat domains disclosed herein are based, in part, on the discovery that co-administration of a DARPin can effectively block the uptake of therapeutic and/or diagnostic agents in the kidney, and therefore can mitigate undesired renal accumulation of such therapeutic and/or diagnostic agents (or of toxic drug moieties comprised in such therapeutic and/or diagnostic agents) administered to subjects. Importantly, the co-administration of designed ankyrin repeat domains according to the invention does not significantly negatively affect the potency of the co-administered therapeutic and/or diagnostic agent.

The designed ankyrin repeat domains or proteins, their use and the methods described herein may contribute to a solution to the problem of nephrotoxic side effects observed for therapeutic and/or diagnostic agents comprising a toxic drug moiety, such as a radionuclide or a cytotoxic molecule, upon administration to a subject, by reducing the accumulation of these agents (or of a drug moiety comprised in such agents) in the kidney.

DARPins are small engineered scaffold proteins (about 14 kDa for a single designed repeat domain) that can be selected to bind a given target protein with high affinity and specificity. Their application in the context of radiopharmaceutical therapy or diagnostics has started to be investigated, for example in a phase I clinical trial involving ^{99m}Tc labeled DARPin for breast cancer imaging (Bragina, O., et al., Journal of Nuclear Medicine 63.4 (2022): 528-535). However, as with other radiopharmaceuticals based on low molecular weight binders, solutions to address nephrotoxicity are required to fully exploit the potential of DARPin-based radiopharmaceuticals.

In one aspect, the invention provides designed ankyrin repeat domains for use in reducing accumulation of a therapeutic and/or diagnostic agent (or of a drug moiety comprised in such agent) in the kidney of a subject treated with said agent, wherein the designed ankyrin repeat domain is administered to the subject in an amount effective to reduce accumulation of said therapeutic and/or diagnostic agent (or of a drug moiety comprised in such agent) in the kidney.

In another aspect, the invention provides a recombinant protein for use in reducing accumulation of a therapeutic and/or diagnostic agent (or of a drug moiety comprised in such agent) in the kidney of a subject treated with said agent, wherein the recombinant protein is administered to the subject in an amount effective to reduce accumulation of said therapeutic and/or diagnostic agent (or of a drug moiety comprised in such agent) in the kidney, and wherein said recombinant protein comprises a designed ankyrin repeat domain for use as described herein.

In another aspect, the invention provides isolated nucleic acids encoding a designed repeat domain for use according to the invention or encoding a recombinant protein for use according to the invention, a recombinant expression vector comprising such nucleic acids, host cells comprising such expression vectors, and pharmaceutical compositions comprising the designed repeat protein for use, recombinant protein for use, nucleic acid and/or recombinant expression vector of the invention and optionally at least one pharmaceutically acceptable carrier or diluent.

In another aspect, the invention provides a method of reducing accumulation of a therapeutic and/or diagnostic agent (or of a drug moiety comprised in such agent) in the kidney of a subject treated with said agent, the method comprising the step of administering to said subject an effective amount of a designed ankyrin repeat domain.

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E):

E1. A designed ankyrin repeat domain for use in reducing accumulation of a therapeutic and/or diagnostic agent in the kidney of a subject treated with said agent, wherein the designed ankyrin repeat domain is administered to the subject in an amount effective to reduce accumulation of said therapeutic and/or diagnostic agent in the kidney.

E2. The designed ankyrin repeat domain for use according to E1, wherein said subject is treated with said agent by systemic administration of said agent.

E3. The designed ankyrin repeat domain for use according to E2, wherein said systemic administration of said agent is by parenteral administration.

E4. The designed ankyrin repeat domain for use according to any one of E1 to E3, wherein the reduction of accumulation of said agent in the kidney is measured between about 1 hour and about 24 hours after treatment of the subject with the agent.

E5. The designed ankyrin repeat domain for use according to any one of E1 to E4, wherein the designed ankyrin repeat domain is administered concomitantly with the agent.

E6. The designed ankyrin repeat domain for use according to any one of E1 to E5, wherein the designed ankyrin repeat domain is administered to the subject at a molar ratio of repeat domain to agent of about 1:1 or higher, about 5:1 or higher, about 20:1 or higher, about 50:1 or higher, about 100:1 or higher, about 500:1 or higher, about 1000:1 or higher, about 1500:1 or higher, about 2000:1 or higher, about 2500:1 or higher, about 3000:1 or higher, or about 3500:1 or higher, about 4000:1 or higher, about 4500:1 or higher, about 5000:1 or higher, about 5500:1 or higher, or about 6000:1 or higher.

E7. The designed ankyrin repeat domain for use according to any one of E1 to E6, wherein the accumulation of said agent in the kidney is reduced by at least 10%, at least 20%, at least 30%, at least 40% or at least 50% as compared to the accumulation of said agent in the kidney of a subject treated with said agent as a control without administration of the designed ankyrin repeat domain.

E8. The designed ankyrin repeat domain for use according to any one of E1 to E7, wherein the designed ankyrin repeat domain does not specifically bind to a target with a dissociation constant (K_{D}) of 10⁻⁷ M or below.

E9. The designed ankyrin repeat domain for use according to any one of E1 to E8, wherein the designed ankyrin repeat domain comprises an N-terminal capping module, a C-terminal capping module and one or more internal repeat module(s).

E10. The designed ankyrin repeat domain for use according to any one of E1 to E9, wherein the designed ankyrin repeat domain comprises:
(a) an N-terminal capping module having the amino acid sequence of SEQ ID NO: 5 or any variant thereof having at least 60%, at least 70%, at least 80% or at least 90% sequence identity with SEQ ID NO: 5, and/or
(b) a C-terminal capping module having the amino acid sequence of SEQ ID NO: 6 or any variant thereof having at least 60%, at least 70%, at least 80% or at least 90% sequence identity with SEQ ID NO: 6, and/or
(c) one or more internal repeat module(s) each independently having the amino acid sequence of SEQ ID NO: 7 or any variant thereof having at least 60%, at least 70%, at least 80% or at least 90% sequence identity with SEQ ID NO: 7.

E11. The designed ankyrin repeat domain for use according to any one of E1 to E10, wherein the designed ankyrin repeat domain comprises:
(a) an N-terminal capping module having the amino acid sequence of SEQ ID NO: 5 or any variant of SEQ ID NO: 5 wherein:
   (i) up to 4 amino acids selected from the randomized positions corresponding to positions 4, 8, 11 and 12 of SEQ ID NO: 5 are substituted by a different amino acid, and
   (ii) up to 15 amino acids selected from the non-randomized positions corresponding to positions 1, 2, 3, 5, 6, 7, 9, 10, 13, 14, 15, 16, 17, 18, 19, 20, 21, 22, 23, 24, 25, 26, 27, 28, 29 and 30 of SEQ ID NO: 5 are substituted by a different amino acid, and/or
(b) a C-terminal capping module having the amino acid sequence of SEQ ID NO: 6 or any variant of SEQ ID NO: 6 wherein:
   (i) up to 5 amino acids selected from the randomized positions corresponding to positions 3, 4, 6, 14 and 15 of SEQ ID NO: 6 are substituted by a different amino acid, and
   (ii) up to 15 amino acids selected from the non-randomized positions corresponding to positions 1, 2, 5, 7, 8, 9, 10, 11, 12, 13, 16, 17, 18, 19, 20, 21, 22, 23, 24, 25, 26, 27 and 28 of SEQ ID NO: 6 are substituted by a different amino acid, and/or
(c) one or more internal repeat module(s) each independently having the amino acid sequence of SEQ ID NO: 7 or any variant of SEQ ID NO: 7 wherein:
   (i) up to 6 amino acids selected from the randomized positions corresponding to positions 3, 4, 6, 11, 14 and 15 of SEQ ID NO: 7 are substituted by a different amino acid, and
   (ii) up to 15 amino acids selected from the non-randomized positions corresponding to positions 1, 2, 5, 7, 8, 9, 10, 12, 13, 16, 17, 18, 19, 20, 21, 22, 23, 24, 25, 26, 27, 28, 29, 30, 31, 32 and 33 of SEQ ID NO: 7 are substituted by a different amino acid.

E12. The designed ankyrin repeat domain for use according to any one of E1 to E11, wherein the designed ankyrin repeat domain has an isoelectric point (pI) in a range between about pH 4.5 and about pH 6.5, preferably between about pH 4.6 and about pH 6.0, and more preferably between about pH 4.7 and about pH 5.5.

E13. The designed ankyrin repeat domain for use according to any one of E1 to E12, wherein the designed ankyrin repeat domain comprises two or more internal repeat modules, preferably two internal repeat modules.

E14. The designed ankyrin repeat domain for use according to E13, wherein the internal repeat modules comprised in the designed ankyrin repeat domain have at least 80%, at least 85%, at least 90%, at least 91%, at least 92%, at least 93%, at least 94%, at least 95%, at least 96%, at least 97%, at least 98%, at least 99% or 100% sequence identity between each other.

E15. The designed ankyrin repeat domain for use according to any one of E1 to E14, wherein the designed ankyrin repeat domain comprises an amino acid sequence selected from the group consisting of (1) SEQ ID NO: 1 and (2) sequences with at least 80% amino acid sequence identity with SEQ ID NO: 1.

E16. The designed ankyrin repeat domain for use according to any one of E1 to E15, wherein the therapeutic and/or diagnostic agent comprises a binding moiety and a drug moiety.

E17. The designed ankyrin repeat domain for use according to E16, wherein said drug moiety is a toxin.

E18. The designed ankyrin repeat domain for use according to E17, wherein said toxin is a radionuclide.

E19. The designed ankyrin repeat domain for use according to E17, wherein said toxin is a cytotoxin.

E20. The designed ankyrin repeat domain for use according to any one of E16 to E19, wherein said binding moiety comprises a designed ankyrin repeat domain with binding specificity for a target.

E21. The designed ankyrin repeat domain for use according to E20, wherein the amino acid sequence of said designed ankyrin repeat domain for use is different from the amino acid sequence of said designed ankyrin repeat domain comprised in said binding moiety.

E22. A recombinant protein for use in reducing accumulation of a therapeutic and/or diagnostic agent in the kidney of a subject treated with said agent, wherein the recombinant protein is administered to the subject in an amount effective to reduce accumulation of said therapeutic and/or diagnostic agent in the kidney, and wherein said recombinant protein comprises the designed ankyrin repeat domain for use according to any one of E1 to E21.

E23. The recombinant protein for use according to E22, wherein the molecular weight of the recombinant protein is 69 kDa or less, 65 kDa or less, 60 kDa or less, 55 kDa or less, 50 kDa or less, 45 kDa or less, 40 kDa or less, 35 kDa or less, or 30 kDa or less.

E24. The designed ankyrin repeat domain for use according to any one of E1 to E21 or the recombinant protein for use according to any one of E22 to E23, wherein the designed ankyrin repeat domain or the recombinant protein is administered to the subject in form of a pharmaceutical composition comprising the designed ankyrin repeat domain or the recombinant protein and optionally at least one pharmaceutically acceptable carrier or diluent.

E25. An isolated nucleic acid encoding the designed ankyrin repeat domain for use according to any one of E1 to E21 or the recombinant protein for use according to any one of E22 to E23.

E26. A recombinant expression vector comprising the nucleic acid according to E25.

E27. A host cell comprising the recombinant expression vector according to E26.

E28. A pharmaceutical composition comprising one or more of: (i) the designed ankyrin repeat domain for use according to any one of E1 to E21, (ii) the recombinant protein for use according to any one of E22 to E23, (iii) the nucleic acid according to E25, and/or (iv) the recombinant expression vector according to E26, and optionally at least one pharmaceutically acceptable carrier or diluent.

E29. A method of reducing accumulation of a therapeutic and/or diagnostic agent in the kidney of a subject treated with said agent, the method comprising the step of administering to the subject an effective amount of a designed ankyrin repeat domain.

E30. The method according to E29, wherein the designed ankyrin repeat domain does not specifically bind to a target with a dissociation constant (K_{D}) of 10⁻⁷ M or below.

E31. The method according to any one of E29 to E30, wherein the designed ankyrin repeat domain comprises an N-terminal capping module, a C-terminal capping module and one or more internal repeat module(s).

E32. The method according to any one of E29 to E31, wherein the designed ankyrin repeat domain comprises two or more internal repeat modules, preferably two internal repeat modules.

E33. The method according to E32, wherein the internal repeat modules comprised in the designed ankyrin repeat domain have at least 80%, at least 85%, at least 90%, at least 91%, at least 92%, at least 93%, at least 94%, at least 95%, at least 96%, at least 97%, at least 98%, at least 99% or 100% sequence identity between each other.

E34. A recombinant protein comprising a designed ankyrin repeat domain, wherein the designed ankyrin repeat domain does not specifically bind to a target with a dissociation constant (K_{D}) of 10⁻⁷ M or below, for use as a medicament.

E35. The recombinant protein for use as a medicament according to E34, wherein the designed ankyrin repeat domain comprises an N-terminal capping module, a C-terminal capping module and one or more internal repeat module(s).

E36. The recombinant protein for use as a medicament according to any one of E34 to E35, wherein the designed ankyrin repeat domain comprises two or more internal repeat modules, preferably two internal repeat modules.

E37. The recombinant protein for use as a medicament according to any one of E34 to E36, wherein the internal repeat modules comprised in the designed ankyrin repeat domain have at least 80%, at least 85%, at least 90%, at least 91%, at least 92%, at least 93%, at least 94%, at least 95%, at least 96%, at least 97%, at least 98%, at least 99% or 100% sequence identity between each other.

E38. The recombinant protein for use as a medicament according to any one of E34 to E37, wherein the designed ankyrin repeat domain comprises:
(a) an N-terminal capping module having the amino acid sequence of SEQ ID NO: 5 or any variant thereof having at least 60%, at least 70%, at least 80% or at least 90% sequence identity with SEQ ID NO: 5, and/or
(b) a C-terminal capping module having the amino acid sequence of SEQ ID NO: 6 or any variant thereof having at least 60%, at least 70%, at least 80% or at least 90% sequence identity with SEQ ID NO: 6, and/or
(c) one or more internal repeat module(s) each independently having the amino acid sequence of SEQ ID NO: 7 or any variant thereof having at least 60%, at least 70%, at least 80% or at least 90% sequence identity with SEQ ID NO: 7.

E39. The recombinant protein for use as a medicament according to any one of E34 to E38, wherein the designed ankyrin repeat domain comprises:
(a) an N-terminal capping module having the amino acid sequence of SEQ ID NO: 5 or any variant of SEQ ID NO: 5 wherein:
   (i) up to 4 amino acids selected from the randomized positions corresponding to positions 4, 8, 11 and 12 of SEQ ID NO: 5 are substituted by a different amino acid, and
   (ii) up to 15 amino acids selected from the non-randomized positions corresponding to positions 1, 2, 3, 5, 6, 7, 9, 10, 13, 14, 15, 16, 17, 18, 19, 20, 21, 22, 23, 24, 25, 26, 27, 28, 29 and 30 of SEQ ID NO: 5 are substituted by a different amino acid, and/or
(b) a C-terminal capping module having the amino acid sequence of SEQ ID NO: 6 or any variant of SEQ ID NO: 6 wherein:
   (i) up to 5 amino acids selected from the randomized positions corresponding to positions 3, 4, 6, 14 and 15 of SEQ ID NO: 6 are substituted by a different amino acid, and
   (ii) up to 15 amino acids selected from the non-randomized positions corresponding to positions 1, 2, 5, 7, 8, 9, 10, 11, 12, 13, 16, 17, 18, 19, 20, 21, 22, 23, 24, 25, 26, 27 and 28 of SEQ ID NO: 6 are substituted by a different amino acid, and/or
(c) one or more internal repeat module(s) each independently having the amino acid sequence of SEQ ID NO: 7 or any variant of SEQ ID NO: 7 wherein:
   (i) up to 6 amino acids selected from the randomized positions corresponding to positions 3, 4, 6, 11, 14 and 15 of SEQ ID NO: 7 are substituted by a different amino acid, and
   (ii) up to 15 amino acids selected from the non-randomized positions corresponding to positions 1, 2, 5, 7, 8, 9, 10, 12, 13, 16, 17, 18, 19, 20, 21, 22, 23, 24, 25, 26, 27, 28, 29, 30, 31, 32 and 33 of SEQ ID NO: 7 are substituted by a different amino acid.

E40. The recombinant protein for use as a medicament according to any one of E34 to E39, wherein the designed ankyrin repeat domain comprises an amino acid sequence selected from the group consisting of (1) SEQ ID NO: 1 and (2) sequences with at least 80% amino acid sequence identity with SEQ ID NO: 1.

### BRIEF DESCRIPTION OF THE FIGURES

FIG. 1: Sequences of DARPins used in the present invention. Randomized positions in the DAPRin library are shown as bold underlined X letter (X) in the consensus sequence row.

FIG. 2: Size exclusion chromatography (SEC) profiles of DARPin01 to DARPin03 (SEQ ID NOS: 1 to 3 respectively) prior to the radiolabeling step. Each DARPin additionally comprises a C-terminal GSGSC tag (SEQ ID NO: 10) and “GS” residues at the N-terminal side. All SEC profiles exhibit a dimeric peak before the main monomeric peak, due to the partial formation of disulfide-linked dimers (C-terminal Cys).

FIG. 3: Graphical summary of the production process of radiolabeled DARPins. DARPins were expressed in E. coli and purified over IMAC (immobilized metal affinity chromatography) and GF (gel filtration). Constructs were cleaved by recombinant TEV protease to cleave off the His-tag. Subsequently, non-cleaved DARPins as well as His-tagged TEV protease were removed by inverse IMAC, flow-through was collected and loaded on a SEC column. Purified DARPins were reduced and coupled with the chelator DTPA. Chelated DARPins were subsequently loaded with radionuclide indium-111 (also referred to as ¹¹¹In).

FIG. 4: Single-trace SPR profiles of DTPA-coupled DARPins against biotinylated full-length human epidermal growth factor receptor 2 (HER2). Plots 1 and 2 show the profiles of the non DTPA-coupled HER2 binders DARPin02 and DARPin03 (SEQ ID NOs: 2 and 3), respectively. Plots 3 and 4 show the profiles of the DTPA coupled counterparts GS-DARPin02-GSGSC-DTPA and GS-DARPin03-GSGSC-DTPA respectively. All analytes (500 nM) were injected in succession for 120 s, dissociation was recorded for 180 s (25 ul/min of PBS-Tween20 (0.005%)). Each injection was followed by a regeneration step with glycine pH2.0 for 60 s. The data was double referenced (control spot and buffer injection) and fitted to a 1:1 Langmuir model.

FIG. 5: Effect of cold DARPin co-injection on kidney uptake of ¹¹¹In-labeled DARPins. Radiolabeled DARPin01, DARPin02 and DARPin03 were injected with (white bars) or without (black bars) a 50-molar-fold excess of cold DARPins (Cold-DARPin01, Cold-DARPin02 and Cold-DARPin03 respectively) into the tail vein of wild-type mice. Data are shown as mean % injected activity/gram of tissue mass (% IA/g). Measures were taken 4 hours after injection. A reduction of 80% (DARPin01), 72% (DARPin02) and 70% (DARPin03) kidney accumulation is observed in the groups co-injected with a cold DARPin as compared to the groups injected with the radiolabeled DARPin only. Error bars show SD.

FIG. 6: Effect of cold DARPin co-injection on kidney uptake of ¹¹¹In-labeled DARPins in HER2-expressing SKOV3ip tumor bearing mice. 6A: Mice were separated into two groups and injected with radiolabeled DARPins (1 mg/kg, approx. 150 KBq) when the tumors reached a volume of approx. 180 mm³ (plot 1) or 360 mm³ (plot 2). Mice in groups 1 and 4 were injected with ¹¹¹In-labeled DARPin02, which has binding specificity for HER2. Mice in groups 2 and 5 were injected with ¹¹¹In-labeled DARPin04, which is a structurally engineered DARPin having a lower isoelectric point (pI) and a lower percentage of basic amino acids compared to DARPin02. Mice in groups 3 and 6 received a co-injection of ¹¹¹In-labeled DARPin04 with a 50-molar-fold excess of cold DARPin (Cold-DARPin01). Cold-DARPin01 is a non-binding DARPin. Data are shown as mean % injected activity/gram of tissue mass (% IA/g). Measures were taken 4 hours after injection. A reduction in kidney accumulation was observed in both cold DARPin-blocked groups (Group 3 and 6) compared to the non-blocked groups (Group 2 and 5) respectively. A reduction of 84.74% (Group2 vs Group1) and 70% (Group5 vs Group4) can be attributed to the engineering of DARPin04 compared to DARPin02. Combining the engineering of DARPin04 with a co-administration of Cold-DARPin01 further significantly decreases the kidney accumulation of radiolabeled DARPin04 by 44.44% (Group3 vs Group2) and 66% (Group6 vs Group5). Error bars show SD. Data were analyzed by using the unpaired, two-tailed Student's t-test. Differences at the 95% confidence level (P<0.05) were statistically significant, *** means P<0.001. 6B: Tumor uptake is shown and remains similar between treatment groups (bars represent pooled tumor sizes, error bars show SEM).

FIG. 7: Effect of cold DARPin co-injection on kidney uptake (plot 1) and tumor uptake (plot 2) of 111In-labeled DARPins in HER2-expressing SKOV3ip tumor bearing mice was tested with DARPin blocker variants. Mice were injected with HER2-specific radiolabeled DARPin02 (1 mg/kg, approx. 150 KBq) at a tumor volume of approx. 350 mm3. Group 1 is a reference group in which 111In-labeled DARPin02 was injected alone. Mice in Groups 2 to 9 received a co-injection of 111In-labeled DARPin02 with a respective 50-molar-fold excess of cold DARPin01 (comprising SEQ ID NO: 1, Group 2), cold DARPin05 (comprising SEQ ID NO: 11, Group 3), cold DARPin06 (comprising SEQ ID NO: 12, Group 4), cold DARPin07 (comprising SEQ ID NO: 13, Group 5), cold DARPin08 (comprising SEQ ID NO: 14, Group 6), cold DARPin09 (comprising SEQ ID NO: 15, Group 7), cold DARPin10 (comprising SEQ ID NO: 16, Group 8) and cold DARPin11 (comprising SEQ ID NO: 17, Group 9). Measures were taken 4 hours after i.v. injection. A significant reduction in kidney accumulation was observed in all cold DARPin-blocked groups (Group 2 to 9) compared to the non-blocked Group 1. More specifically, a reduction of 57.09% (Group 2 vs Group 1), 39.99% (Group 3 vs Group 1), 37.15% (Group 4 vs Group 1), 33.30% (Group 5 vs Group 1), 52.32% (Group 6 vs Group 1), 41.51% (Group 7 vs Group 1), 21.35% (Group 8 vs Group 1) and 26.20% (Group 9 vs Group 1) was observed. Tumor uptake (plot 2) remains similar between treatment groups. Data are shown as mean % injected activity/gram of tissue mass (% IA/g). Error bars show SD. Data were analyzed by ANOVA and Dunnett's test. Statistically significant changes are displayed with *p<0.05, **p<0.01, ***p<0.001, ****p<0.0001 and ns p>0.05.

FIG. 8: Effect of cold DARPin co-injection (Cold-DARPin01 comprising SEQ ID NO: 1, Group 2) on kidney uptake (8A) and tumor uptake (8B) of 111In-labeled DARPin04 in HER2-expressing SKOV-3 tumor (200-500 mm3) bearing mice, compared to co-injection of 111In-labeled DARPin04 with comparative compound 1 (alpha-1-microglobulin variant of SEQ ID NO: 18, Group 3) or comparative compound 2 (Gelofusine, Group 4). Group 1 is the reference treatment in which 111In-labeled DARPin04 was injected alone. Detailed dosing is shown in Table 14. Measures were taken 4 hours after injection. Co-injection with Cold-DARPin01 (Group 2) results in the highest kidney accumulation reduction (i.e 67.5% reduction) compared to the co-injection with comparative compound 1 (Group 3, 50.7% reduction) and comparative compound 2 (Group 4, 28.4% reduction). Tumor uptake remains similar between treatment groups. Data are shown as mean % injected activity/gram of tissue mass (% IA/g). Error bars show SD.

### DETAILED DESCRIPTION OF THE INVENTION

The inventors of the present invention have surprisingly discovered that co-administration of non-radiolabeled (or cold) DARPins could significantly reduce the kidney uptake of radiolabeled agents (such as, e.g., radiolabeled DARPins) (or of drug moieties comprised in such agents) and hence the underlying radioactivity accumulation in kidneys, both in cases where the cold DARPin is structurally identical or different from the radiolabeled agent. In case of a DARPin as the agent, the agent and the cold DARPin can have different binding specificities, such as, e.g., no binding specificity for the cold DARPin but target binding specificity for the radiolabeled DARPin.

Based on these observations, the DARPins and uses and methods described herein are envisaged to be applied with a broad spectrum of radiolabeled or non-radiolabeled therapeutic and/or diagnostic agents which are expected to enter into renal tubular cells, such as by megalin/cubulin receptor complex mediated endocytosis, hereby exerting a potential nephrotoxicity.

Methods to reduce kidney accumulation and mitigating agents in this context are highly desirable since renal accumulation of a toxic drug moiety in the kidneys leads to nephrotoxicity, which often constrains the use of radiolabeled agents in therapy or diagnostics. Accordingly, the designed ankyrin repeat domains and proteins for use and methods to reduce kidney uptake provided herein may solve a prominent problem in therapeutic and/or diagnostic applications involving drug-moiety linked agents for instance in the field of nuclear medicine such as in radiopharmaceutical therapy or diagnostic of cancer, or agents such as cytotoxic-conjugated proteins for cancer therapy.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry described herein are those well-known and commonly used in the art.

The terms “comprising”, “having”, “including” and “containing” are to be construed as open-ended terms unless otherwise noted. If aspects of the invention are described as “comprising” a feature, embodiments also are contemplated “consisting of” or “consisting essentially of” the feature. The use of any and all examples, or exemplary language (e.g., “such as”) provided herein, is intended merely to better illustrate the disclosure and does not pose a limitation on the scope of the disclosure unless otherwise claimed. No language in the specification should be construed as indicating any non-claimed element as essential to the practice of the disclosure. The term “about” as used herein is equivalent to +10% of a given numerical value, unless otherwise stated.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

The term “nucleic acid” refers to a polynucleotide molecule, which may be a ribonucleic acid (RNA) or deoxyribonucleic acid (DNA) molecule, either single stranded or double stranded, and includes modified and artificial forms of DNA or RNA. A nucleic acid may either be present in isolated form or be comprised in recombinant nucleic acid molecules or vectors.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g. by a disulfide bond between two polypeptides. A part of a protein, which individually has, or is able to acquire, a defined three-dimensional arrangement by forming secondary and/or tertiary structure, is termed “protein domain”. Such protein domains are well known to the practitioner skilled in the art.

The term “recombinant” as used in recombinant protein, recombinant polypeptide and the like, means that said protein or polypeptide is produced by the use of recombinant DNA technologies well known to the practitioner skilled in the art. For example, a recombinant DNA molecule (e.g. produced by gene synthesis) encoding a polypeptide can be cloned into a bacterial expression plasmid (e.g. pQE30, QIAgen), yeast expression plasmid, mammalian expression plasmid, or plant expression plasmid, or a DNA enabling in vitro expression. If, for example, such a recombinant bacterial expression plasmid is inserted into appropriate bacteria (e.g. Escherichia coli), these bacteria can produce the polypeptide(s) encoded by this recombinant DNA. The correspondingly produced polypeptide or protein is called a recombinant polypeptide or recombinant protein.

In the context of the present invention, the term “polypeptide” relates to a molecule consisting of a chain of multiple, i.e. two or more, amino acids linked via peptide bonds. Preferably, a polypeptide consists of more than eight amino acids linked via peptide bonds. The term “polypeptide” also includes multiple chains of amino acids, linked together by S-S bridges of cysteines. Polypeptides are well-known to the person skilled in the art.

The term “target” refers to an individual molecule such as a nucleic acid, a polypeptide or protein, a carbohydrate, or any other naturally or non-naturally occurring molecule or moiety, including any part of such individual molecule, or complexes of two or more of such molecules. The target may also be a whole cell or a tissue sample. Preferably, the target is a naturally occurring or non-natural polypeptide or a polypeptide containing chemical modifications, for example modified by natural or non-natural phosphorylation, acetylation, or methylation.

Patent application WO2002020565 and Forrer et al., 2003 (Forrer, P., Stumpp, M. T., Binz, H. K., Pluckthun, A., 2003. FEBS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers.

The term “repeat domain” refers to a protein domain comprising two or more consecutive repeat modules as structural units, wherein said repeat modules have structural and sequence homology. Preferably, a repeat domain also comprises an N-terminal and/or a C-terminal capping module. For clarity, a capping module can be a repeat module. Such repeat domains, repeat modules, and capping modules, sequence motifs, as well as structural homology and sequence homology are well known to the practitioner in the art from examples of ankyrin repeat domains (Binz et al., J. Mol. Biol. 332, 489-503, 2003; Binz et al., 2004, loc. cit.; WO2002020565; WO2012069655), leucine-rich repeat domains (WO2002020565), tetratricopeptide repeat domains (Main, E. R., Xiong, Y., Cocco, M. J., D'Andrea, L., Regan, L., Structure 11 (5), 497-508, 2003), and armadillo repeat domains (WO2009040338). It is further well known to the practitioner in the art that such repeat domains are different from proteins comprising repeated amino acid sequences, where every repeated amino acid sequence is able to form an individual domain (for example FN3 domains of Fibronectin). The repeat domains can be binding domains.

The term “ankyrin repeat domain” refers to a repeat domain comprising two or more consecutive ankyrin repeat modules as structural units, wherein said ankyrin repeat modules have structural and sequence homology.

The term “designed” as used in designed repeat protein, designed repeat domain and the like refers to the property that such repeat proteins and repeat domains, respectively, are man-made and do not occur in nature. The binding domains described herein are designed repeat domains. Preferably, a designed repeat domain described herein is a designed ankyrin repeat domain.

The term “repeat modules” refers to the repeated amino acid sequence and structural units of the designed repeat domains, which are originally derived from the repeat units of naturally occurring repeat proteins. Each repeat module comprised in a repeat domain is derived from one or more repeat units of a family or subfamily of naturally occurring repeat proteins, preferably the family of ankyrin repeat proteins. Furthermore, each repeat module comprised in a repeat domain may comprise a “repeat sequence motif” deduced from homologous repeat modules obtained from repeat domains selected on a target and having the same target specificity. A repeat module as used in the present invention encompasses internal repeat modules and capping modules such as N-terminal and C-terminal capping modules. An “internal repeat module” refers to a repeat module that is flanked by two repeat modules. In other words, an internal repeat module is N-terminally flanked by one repeat module and C-terminally flanked by another repeat module.

Accordingly, the term “ankyrin repeat module” refers to a repeat module, which is originally derived from the repeat units of naturally occurring ankyrin repeat proteins. Ankyrin repeat proteins are well known to the person skilled in the art. Designed ankyrin repeat proteins have been described previously; see, e.g., International Patent Publication WO2002020565, WO2010060748, WO2011135067, WO2012069654, WO2012069655, WO2014001442, WO2014191574, WO2014083208, WO2016156596, and WO2018054971, all of which are incorporated by reference in their entireties. Typically, an ankyrin repeat module comprises about 31 to 33 amino acid residues that form two alpha helices, separated by loops.

Repeat modules may comprise positions with amino acid residues which have not been randomized in a library for the purpose of selecting target-specific repeat domains (“non-randomized positions” or “fixed positions” used interchangeably herein) and positions with amino acid residues which have been randomized in the library for the purpose of selecting target-specific repeat domains (“randomized positions”). Non-randomized positions comprise framework residues and may also comprise target interaction residues. The randomized positions comprise target interaction residues. “Have been randomized” means that two or more amino acids were allowed at an amino acid position of a repeat module, for example, wherein any of the usual twenty naturally occurring amino acids were allowed, or wherein most of the twenty naturally occurring amino acids were allowed, such as amino acids other than cysteine, or amino acids other than glycine, cysteine and proline. For the purpose of this patent application, positions 4, 8, 11 and 12 numbered relative to SEQ ID NO: 5; positions 3, 4, 6, 14 and 15 numbered relative to SEQ ID NO: 6; and positions 3, 4, 6, 11, 14 and 15 numbered relative to SEQ ID NO: 7 are randomized positions of the ankyrin repeat modules described herein. The term “non-randomized positions” does not include the positions within the designed ankyrin repeat domain that correspond to the randomized positions.

The term “repeat sequence motif” refers to an amino acid sequence, which is deduced from one or more repeat modules. Preferably, said repeat modules are from repeat domains having binding specificity for the same target. Such repeat sequence motifs comprise framework residue positions and target interaction residue positions. Said framework residue positions correspond to the positions of framework residues of the repeat modules. Likewise, said target interaction residue positions correspond to the positions of target interaction residues of the repeat modules. Repeat sequence motifs comprise non-randomized positions and randomized positions.

The term “repeat unit” refers to amino acid sequences comprising sequence motifs of one or more naturally occurring proteins, wherein said “repeat units” are found in multiple copies, and exhibit a defined folding topology common to all said motifs determining the fold of the protein. Examples of such repeat units include leucine-rich repeat units, ankyrin repeat units, armadillo repeat units, tetratricopeptide repeat units, HEAT repeat units, and leucine-rich variant repeat units.

A residue or amino acid residue refers to an amino acid comprised in a peptide. The term “target interaction residues” refers to amino acid residues of a repeat module, which contribute to the direct interaction with a target. Such contribution of a residue can be tested, e.g., in a binding assay, for example in a mutagenesis study performed to identify residues required, sufficient, and/or necessary for a repeat domain to bind a target with its original binding affinity or quantity (i.e. its binding affinity or quantity in the absence of any mutations). Target interaction residues can also be determined by structural analyses of a repeat domain bound to a target.

The term “framework residues” refers to amino acid residues of a repeat module, which contribute to the folding topology, i.e. which contribute to the fold of said repeat module or which contribute to the interaction with a neighboring module. Such contribution may be the interaction with other residues in the repeat module, or the influence on the polypeptide backbone conformation as found in α-helices or β-sheets, or the participation in amino acid stretches forming linear polypeptides or loops.

Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

The term “binding specificity”, “has binding specificity for a target”, “specifically binding to a target”, “binding to a target with high specificity”, “specific for a target” or “target specificity” and the like means that a binding protein or binding domain binds to a target with a lower dissociation constant (i.e. it binds with higher affinity) than it binds to an unrelated protein such as the E. coli maltose binding protein (MBP). Preferably, the dissociation constant (“K_{D}”) for the target is at least 10²; more preferably, at least 10³; more preferably, at least 10⁴; or more preferably, at least 10⁵ times lower than the corresponding dissociation constant for MBP. Methods to determine dissociation constants of protein-protein interactions, such as surface plasmon resonance (SPR) based technologies (e.g. SPR equilibrium analysis) or isothermal titration calorimetry (ITC) are well known to the person skilled in the art. The measured K_{D} values of a particular protein-protein interaction can vary if measured under different conditions (e.g., salt concentration, pH). Thus, measurements of K_{D} values are preferably made with standardized solutions of protein and a standardized buffer, such as PBS.

Binding of any molecule to another is governed by two forces, namely the association rate (kₒₙ) and the dissociation rate (k_{off}). The affinity of any binder [B] to a target [T] can then be expressed by the equilibrium dissociation constant K_{D}, which is the quotient of k_{off}/kₒₙ.

$$[ B ] + [ T ] {{⇌}_{{k}_{off}}}^{{k}_{on}} [ BT ]$$

kₒₙ is a second-order rate constant of the binding reaction, with the unit M⁻¹ s⁻¹, whereas the dissociation reaction k_{off} is a first-order rate constant with the unit s⁻¹. From this it becomes clear that the association reaction depends on the concentration of the reactants, whereas the dissociation is independent of the concentration, following a simple exponential decay function.

A variety of methods of measuring binding affinity are known in the art, any of which can be used for purposes of the present invention. For example, as exemplified herein, the binding affinity of a particular binding moiety to a drug molecule target can be expressed as K_{D} value, which refers to the dissociation constant of the binding moiety and the drug molecule target. K_{D} is the ratio of the rate of dissociation, also called the “off-rate (k_{off})”, to the association rate, or “on-rate (kₒₙ)”. Thus, K_{D} equals k_{off}/kₒₙ and is expressed as a molar concentration (M), and the smaller the K_{D}, the stronger the affinity of binding.

K_{D} values can be determined using any suitable method. One exemplary method for measuring K_{D} is surface plasmon resonance (SPR) (see, e.g., Nguyen et al. Sensors (Basel). 2015 May 5; 15(5):10481-510). K_{D} value may be measured by SPR using a biosensor system such as a BIACORE® system. BIAcore kinetic analysis comprises, e.g., analysing the binding and dissociation of an antigen from chips with immobilized molecules (e.g., molecules comprising epitope binding domains), on their surface. Another method for determining the K_{D} of a protein is by using Bio-Layer Interferometry (see, e.g., Shah et al. J Vis Exp. 2014; (84): 51383). A K_{D} value may be measured using OCTET® technology (Octet QKe system, ForteBio). Alternatively, or in addition, a KinExAR (Kinetic Exclusion Assay) assay, available from Sapidyne Instruments (Boise, Id.) can also be used. Any method suitable for assessing the binding affinity between two binding partners is encompassed herein. Surface plasmon resonance (SPR) is particularly preferred. Most preferably, the K_{D} values are determined in PBS and by SPR.

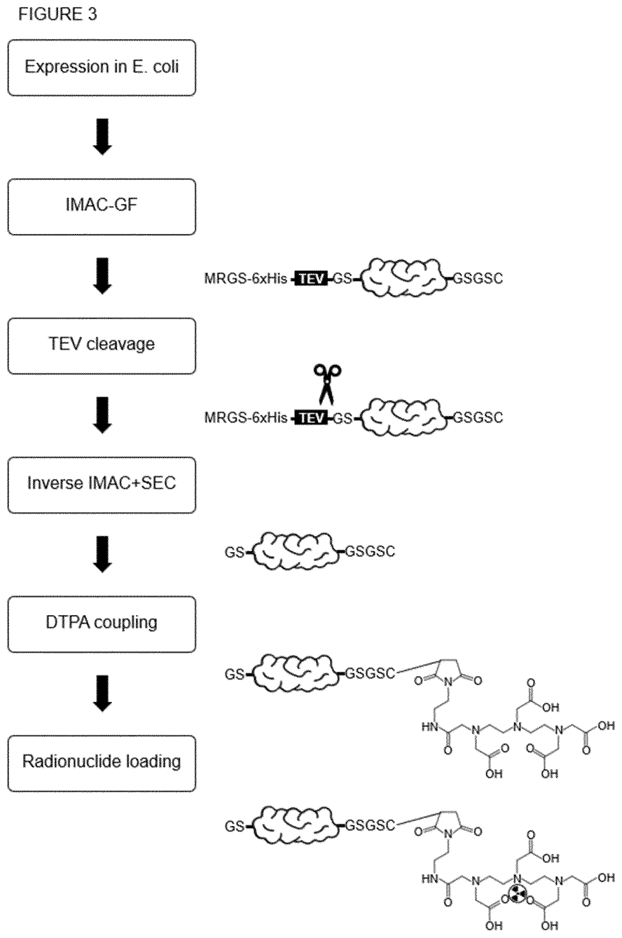

The term “Isoelectric point” or “pI” refers to the pH value at which a macromolecule such as a protein carries no net electrical charge. In proteins there may be many charged groups, and at the isoelectric point the sum of all these charges is zero. At a pH above the isoelectric point the overall net charge of the polypeptide will be negative, whereas at pH values below the isoelectric point the overall net charge of the polypeptide will be positive. Isoelectric points can be determined experimentally or can be calculated for polypeptides based on the primary sequence. The skilled person is aware of methods to determine the isoelectric point of a protein. Most commonly, the isoelectric point of a protein is computed based on the amino acid sequence of the protein. Numerous tools are available online that allow computing the isoelectric point of a protein. One such preferred tool is “ExPASy Compute pI/Mw” (https://web.expasy.org/compute_pi/); see Protein Identification and Analysis Tools on the ExPASy Server; Gasteiger E., Hoogland C., Gattiker A., Duvaud S., Wilkins M. R., Appel R. D., Bairoch A.; (In) John M. Walker (ed): The Proteomics Protocols Handbook, Humana Press (2005), pp. 571-607. This “ExPASy Compute pI/Mw” tool is preferably used for the determination of the pI of ankyrin repeat domains described herein. Any N-terminal or C-terminal tags comprising one or more amino acids which may be fused to a repeat domain for production or other purposes, as well as any N-terminal or C-terminal peptide linkers are not considered for computing the pI of the repeat domains described herein. Such tags or linkers are well known in the art and include for instance the His6-TEV tag of SEQ ID NO: 8 (N-terminal), the GS residues (N-terminal), the MRGSHis6GS tag of SEQ ID NO: 9 (N-terminal) and the GSGSC tag of SEQ ID NO: 10 (C-terminal), as shown for instance in FIG. 3.

The term “basic amino acid” refers to a hydrophilic amino acid having a positively charged side chains at physiological pH. From the 20 common amino acids, His (H), Arg (R), and Lys (K) are basic amino acids. The term “acidic amino acid” refers to a hydrophilic amino acid having a negatively charged side chains at physiological pH. From the 20 common amino acids, Asp (D) and Glu (E) are acidic amino acids. Basic and acidic amino acids can also be collectively referred to as charged amino acids, since at physiological pH their side chains are ionized. “Neutral amino acid” refers to amino acids which are neither basic nor acidic, and are hence effectively non-ionized under physiological conditions. From the 20 common amino acids, Gly (G), Ala (A), Pro (P), Val (V), Leu (L), Ile (I), Met (M), Phe (F), Tyr (T), Trp (W), Ser(S), Thr (T), Cys (C), Asn (N) and Gln (Q) are neutral amino acids. Such considerations are well known to a skilled person in the art.

The term “binding moiety” refers to any molecule capable of specifically binding a target molecule. Binding moieties include, for example, antibodies, antibody fragments, aptamers, peptides (e g, Williams et at, J Biol Chem 266:5182-5190 (1991)), alternative scaffolds, antibody mimics, repeat proteins, e.g, designed ankyrin repeat proteins, receptor proteins and any other naturally occurring interaction partners of the target molecule, and can comprise natural proteins and proteins modified or genetically engineered, e.g., to include non-natural residues and/or to lack natural residues.

The term “drug moiety” refers to a chemical moiety that is linked or is suitable for linkage to a protein and includes any therapeutic or diagnostic agent that has desired therapeutic and/or diagnostic properties, such as for example an anti-cancer, anti-inflammatory or anti-infective agent (e.g., anti-fungal, antibacterial, anti-parasitic, anti-viral). Examples of anti-cancer agents include a toxin or a cytotoxin. Drug moiety as used herein encompasses the terms “therapeutic moiety” and “diagnostic moiety”. Such drug moieties can be linked to a protein, such as, e.g., a repeat domain or a repeat protein, using methods available in the art, or for instance as described in Example 1.

The term “therapeutic moiety” refers to a chemical moiety that can function as a therapeutic agent (or perform a therapeutic function), such as for a treatment of a disease or disorder when administered to or otherwise provided to a patient or subject.

The term “diagnostic moiety” refers to a chemical moiety that can function as a diagnostic agent (or perform a diagnostic function), such as for a diagnosis of a disease or disorder when administered to or otherwise provided to a patient or subject.

The term “linked” or “linkage” refers to any covalent or non-covalent linkage between a chemical moiety and a protein such as a designed repeat domain or a designed repeat protein.

The term “toxin” refers to any agent that is detrimental to the growth, proliferation and/or survival of cells and may act to reduce, inhibit, kill and/or destroy a cell or malignancy. This term encompasses for instance a radionuclide, which may be toxic because of its radioactivity, and a cytotoxic agent.

The term “cytotoxic agent” or “cytotoxin” refers to a substance that causes cell death or toxicity primarily by interfering with a cell's vital processes, such as for example gene expression activity, DNA replication, cell division, and/or cell survival. Non-limiting examples of cytotoxins include chemotherapeutic agents, mitotic inhibitors, growth inhibitory agents, enzymes and fragments thereof such as nucleolytic enzymes, antibiotics, toxins or enzymatically active toxins of bacterial, fungal, plant or animal origin, auristatins, calicheamicins, maytansinoids and camptothecin analogues. Further non-limiting examples of cytotoxins are cytotoxins which can be used in antibody-drug conjugates as described e.g. in Drago, Joshua Z., Shanu Modi, and Sarat Chandarlapaty. Nature Reviews Clinical Oncology 18.6 (2021).

The term “radionuclide” or “radioisotope” refers to isotopes of natural or artificial origin with an unstable neutron to proton ratio that disintegrates with the emission of corpuscular (i.e. protons (alpha-radiation) or electrons (beta-radiation)) or electromagnetic radiation (gamma-radiation). In other words, radionuclides undergo radioactive decay. Such radionuclides include, without limitation, ⁹⁴Tc, ^{99m}Tc, ⁹⁰In, ¹¹¹In, ⁶⁷Ga, ⁶⁸Ga, ⁸⁶Y, ⁹⁰Y, ¹⁷Lu, ¹⁵¹Tb, ²³Ra, ¹⁸⁶Re, ¹⁸Re, ⁶⁴Cu, ⁶⁷Cu, ⁵⁵Co, ⁵⁷Co, ⁴³Sc, ⁴⁴Sc, ⁴⁷Sc, ²³⁵Ac, ²¹³Bi, ²¹²Bi, ²¹²Pb, ²²⁷Th, ¹⁵³Sm, ¹⁶⁶Ho, ¹⁵²Gd, ¹⁵³Gd, ¹⁵⁷Gd, ²²⁵Ac or ¹⁶⁶Dy. The choice of suitable radionuclides may depend on the chemical structure and chelating capability of the chelating agent, and the intended application of the resulting drug (e.g. diagnostic vs. therapeutic).

The terms “chelator” or “chelating agent” refer to polydentate (multiple bonded) ligands capable of forming two or more separate coordinate bonds with (“coordinating”) a central (metal) ion. Specifically, such molecules or molecules sharing one electron pair may also be referred to as “Lewis bases”. The central (metal) ion is usually coordinated by two or more electron pairs to the chelating agent. Usually, the electron pairs of a chelating agent forms coordinate bonds with a single central (metal) ion; however, in certain examples, a chelating agent may form coordinate bonds with more than one metal ion, with a variety of binding modes being possible. The terms “coordinating” and “coordination” refer to an interaction in which one multi-electron pair donor coordinatively bonds (“is coordinated”) to, i.e. shares two or more unshared pairs of electrons with, one central (metal) ion. The chelating agent is preferably chosen based on its ability to coordinate the desired central (metal) ion, usually a radionuclide as specified herein.

The term “physiological conditions” refers to conditions normally present in a mammalian body. Thus, for example for humans, physiological conditions mean a pH between 7.35 and 7.45, with the average at 7.40, and a temperature between 36.1° C. and 37.2° C., with the average at 37° C.

The term “subject”, as used herein generally includes humans and non-human animals and preferably mammals (e.g. non-human primates, including marmosets, tamarins, spider monkeys, owl monkeys, vervet monkeys, squirrel monkeys, and baboons, macaques, chimpanzees, orangutans, gorillas, cows, horses, sheep, pigs, chicken, cats, dogs, mice, rat, rabbits, guinea pigs etc.), including chimeric and transgenic animals and disease models. In the context of the present invention, the term “subject” preferably refers to a non-human primate or a human, most preferably a human.

### Designed Ankyrin Repeat Domains for Use According to the Invention

In a first aspect, the invention provides a designed ankyrin repeat domain for use in reducing accumulation of a therapeutic and/or diagnostic agent (or of a drug moiety comprised in such agent) in the kidney of a subject treated with said agent, wherein the designed ankyrin repeat domain is administered to the subject in an amount effective to reduce accumulation of said therapeutic and/or diagnostic agent (or of a drug moiety comprised in such agent) in the kidney.

The therapeutic and/or diagnostic agents encompassed in the uses of the invention include any agent that has a desired therapeutic and/or diagnostic property and that can be administered to a subject in need of a therapy and/or a diagnosis. Therapeutic and/or diagnostic agents used in the field of nuclear medicine such as in radiopharmaceutical therapy or diagnosis of cancer, and agents such as cytotoxic-conjugated proteins for cancer therapy are preferred. A radiopharmaceutical generally refers to any radioactive compound that can be used as a therapeutic and/or diagnostic agent. Radiopharmaceuticals may comprise a radionuclide and a binding moiety so that the radionuclide is delivered to a target site, e.g. the tumor tissue, in a targeted manner. Radiopharmaceutical agents having both therapeutic and diagnostic properties may also be referred to as theranostic agents.

Accordingly, in some embodiments of the uses described herein, said therapeutic and/or diagnostic agent comprises a binding moiety. In some embodiments, said binding moiety comprises a small organic molecule, a peptide, a monoclonal antibody variant or fragment, or an alternative scaffold as further described herein. In some embodiments, said binding moiety binds to a target with a dissociation constant (K_{D}) of about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less.

Alternative scaffolds include any polypeptides or proteins comprising a binding domain that is capable of binding a target and that is not derived from an antibody or immunoglobulin molecule. The binding domain of alternative scaffolds may comprise or may be derived from a variety of different polypeptide or protein structures. Alternative scaffolds include, but are not limited to, adnectins (monobodies), affibodies, affilins, affimers and aptamers, affitins, alphabodies, anticalins, armadillo repeat protein-based scaffolds, atrimers, avimers, ankyrin repeat protein-based scaffolds (such as DARPin proteins), fynomers, knottins, and Kunitz domain peptides. Alternative scaffolds are described, e.g., in Yu et al., Annu Rev Anal Chem (Palo Alto Calif). 2017 Jun. 12; 10(1): 293-320. doi:10.1146/annurevanchem-061516-045205.

Adnectins are originally derived from the tenth extracellular domain of human fibronectin type III protein (10Fn3). The fibronectin type III domain has 7 or 8 beta strands, which are distributed between two beta sheets, which themselves pack against each other to form the core of the protein, and further contain loops (analogous to CDRs), which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). Because of this structure, this non-antibody scaffold mimics antigen binding properties that are similar in nature and affinity to those of antibodies. These scaffolds can be used in a loop randomization and shuffling strategy in vitro that is similar to the process of affinity maturation of antibodies in vivo.

Affibody affinity ligands are composed of a three-helix bundle based on the scaffold of one of the IgG-binding domains of Protein A, which is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, but are considerably smaller, having a molecular weight of around 6 kDa, compared to around 150 kDa for antibodies. Despite the size difference, the binding site of affibody molecules has similarity to that of an antibody.

Affilins are synthetic antibody mimetics that are structurally derived from human ubiquitin (historically also from gamma-B crystallin). Affilins consists of two identical domains with mainly beta sheet structure and a total molecular mass of about 20 kDa. They contain several surface-exposed amino acids that are suitable for modification. Affilins resemble antibodies in their affinity and specificity to antigens but not in structure.

Affimers are a type of peptide aptamer, having a structure known as SQT (Stefin A quadruple mutant-Tracy). Aptamers and affimers are short peptides responsible for affinity binding with an inert and rigid protein scaffold for structure constraining in which both N- and C-termini of the binding peptide are embedded in the inert scaffold.

Affitins are variants of the DNA binding protein Sac7d that are engineered to obtain specific binding affinities. Sac7d is originally derived from the hyperthermophile archaea Sulfolobus acidocaldarius and binds with DNA to prevent it from thermal denaturation. Affitins are commercially known as Nanofitins.

Alphabodies are small (approximately 10 kDa) proteins that are engineered to bind to a variety of antigens and are therefore antibody mimetics. The alphabody scaffold is computationally designed based on coiled-coil structures. The standard alphabody scaffold contains three α-helices, composed of four heptad repeats (stretches of 7 residues) each, connected via glycine/serine-rich linkers. The standard heptad sequence is “IAAIQKQ”. Alphabodies' ability to target extracellular and intracellular proteins in combination with their high binding affinities may allow them to bind to targets that cannot be reached with antibodies.

Anticalins are a group of binding proteins with a robust and conservative β-barrel structure found in lipocalins. Lipocalins are a class of extracellular proteins comprising one peptide chain (150-190 amino acids) that is in charge of recognition, storage, and transport of various biological molecules such as signaling molecules.

Armadillo repeat protein-based scaffolds are abundant in eukaryotes and are involved in a broad range of biological processes, especially those related to nuclear transport. Armadillo repeat protein-based scaffolds usually consist of three to five internal repeats and two capping elements. They also have a tandem elongated super helical structure that enables binding with their corresponding peptide ligands in an extended conformation.

Atrimers are a scaffold derived from a trimeric plasma protein known as tetranectin, belonging to a family of C-type lectins consisting of three identical units. The structure of the C-type lectin domain (CTLD) within the tetranectin has five flexible loops that mediate interaction with targeting molecules.

Avimers are derived from natural A-domain containing proteins such as HER3 and consist of a number of different “A-domain” monomers (2-10) linked via amino acid linkers. Avimers can be created that can bind to the target antigen using the methodology described in, for example, U.S. Patent Application Publication Nos. 2004/0175756; 2005/0053973; 2005/0048512; and 2006/0008844.

Fynomers are small globular proteins (approximately 7 kDa) that evolved from amino acids 83-145 of the Src homology domain 3 (SH3) of the human Fyn tyrosine kinase. Fynomers are attractive binding molecules due to their high thermal stability, cysteine-free scaffold, and human origin, which reduce potential immunogenicity.

Knottins, also known as cysteine knot miniproteins, are typically proteins 30 amino acids in length comprising three antiparallel β-sheets and constrained loops laced by a disulfide bond, which creates a cysteine knot. This disulfide bond confers high thermal stability making knottins attractive antibody mimetics.

Kunitz domain peptides or Kunitz domain inhibitors are a class of protease inhibitors with irregular secondary structures containing ~60 amino acids with three disulfide bonds and three loops that can be mutated without destabilizing the structural framework.

In preferred embodiments of the uses described herein, said binding moiety comprises a designed ankyrin repeat domain with binding specificity for a target. In other embodiments, said binding moiety consists of a designed ankyrin repeat domain with binding specificity for a target.

Designed ankyrin repeat domains are structural units of designed ankyrin repeat proteins. Designed ankyrin repeat proteins comprising only a single designed ankyrin repeat domain are small proteins (~14 kDa), and the possibility of combining two, three, four, five or more designed ankyrin repeat domains in one protein make designed ankyrin repeat proteins ideal agonistic, antagonistic and/or inhibitory drug candidates. Furthermore, such ankyrin repeat proteins can be engineered to carry various effector functions, e.g. cytotoxic agents or half-life extending agents, enabling completely new drug formats.

Designed repeat protein libraries, including designed ankyrin repeat protein libraries (WO2002020565; Binz et al., Nat. Biotechnol. 22, 575-582, 2004; Stumpp et al., Drug Discov. Today 13, 695-701, 2008; U.S. Pat. No. 9,458,211), can be used for the selection of target-specific designed repeat domains that bind to their target with high affinity. Such target-specific designed repeat domains in turn can be used as valuable components of recombinant binding proteins or (radio) pharmaceuticals for the treatment and/or diagnosis of diseases.

As used herein, the term “repeat module” encompasses internal repeat modules and terminal repeat modules (N-terminal and C-terminal capping modules). 27 of the 33 amino acid positions of typical internal repeat modules are highly conserved, whereas the other 6 amino acid positions are less conserved and to the most part responsible for the specific interaction of the ankyrin repeat domain with its target (Binz et al. 2003, loc. cit.). The paratope of the ankyrin repeat domain is formed by the continuous surface formed largely by these variable positions of the internal repeat modules and sometimes also the capping repeat modules. DARPins also encompass proteins which comprise multiple designed ankyrin repeat domains linked together by appropriate linkers. Such linkers are known to the person skilled in the art.

In some embodiments of the uses described herein, said therapeutic and/or diagnostic agent comprises a drug moiety and/or a binding moiety. Accordingly, in some embodiments of the uses described herein, said therapeutic and/or diagnostic agent comprises a drug moiety. In some embodiments of the uses described herein, said therapeutic and/or diagnostic agent comprises a binding moiety.

Preferably, in some embodiments of the uses described herein, said therapeutic and/or diagnostic agent comprises a drug moiety and a binding moiety. In particular embodiments, said drug moiety is covalently or non-covalently linked to the binding moiety. In other particular embodiments of the uses described herein, said drug moiety is linked to said binding moiety by a chelator. Appropriate chelators may be selected depending on the nature of the binding and drug moieties. In a particular embodiment of the uses described herein, said chelator is diethylenetriaminepentaacetic acid (DTPA).

In some embodiments of the uses described herein, said drug moiety is a therapeutic and/or diagnostic moiety. In preferred embodiments of the uses described herein, said drug moiety is a radionuclide. The choice of said drug moiety may depend on the intended purpose of the of the agent (e.g. diagnostic vs. therapeutic).

In particular embodiments of the uses described herein, said drug moiety is a therapeutic moiety. In some embodiments of the uses described herein, said therapeutic moiety is a toxin. In more particular embodiments of the uses described herein, said therapeutic moiety is a radionuclide as defined herein. In other more particular embodiments of the uses described herein, said therapeutic moiety is a cytotoxin as defined herein.

In particular embodiments of the uses described herein, said drug moiety is a diagnostic moiety. In a more particular embodiment of the uses described herein, said diagnostic moiety is a fluorophore, a chromophore, an imaging agent or a radionuclide.

Accordingly, in a preferred embodiment of the uses described herein, said therapeutic and/or diagnostic agent comprises a binding moiety and a drug moiety, wherein said binding moiety comprises or consists of a designed ankyrin repeat domain with binding specificity for a target. In particular embodiments of the uses described herein, said designed ankyrin repeat domain is covalently or non-covalently linked to said drug moiety. In particular embodiments of the uses described herein, said designed ankyrin repeat domain is linked to said drug moiety by a chelator. In particular embodiments of the uses described herein, said chelator is diethylenetriaminepentaacetic acid (DTPA). In particular embodiments of the uses described herein, said drug moiety is a radionuclide. In some particular embodiments of the uses described herein, said radionuclide is indium-111. In other particular embodiments of the uses described herein, said drug moiety is a cytotoxin.

In further embodiments of the uses described herein, said ankyrin repeat domain comprised in said binding moiety binds specifically to a target. In some embodiments, said ankyrin repeat domain comprised in said binding moiety binds to said target with a dissociation constant (K_{D}) of about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less.

In some preferred embodiments, the amino acid sequence of said designed ankyrin repeat domain for use is different from the amino acid sequence of the designed ankyrin repeat domain comprised in said binding moiety. In some embodiments, the amino acid sequence of said designed ankyrin repeat domain for use differs in sequence identity from the amino acid sequence comprised in said binding moiety by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or by 100%, In some preferred embodiments, the designed ankyrin repeat domain for use is not comprised in said binding moiety.

The timepoint at which the accumulation of said agent in the kidney is determined may depend on the chemical properties of the agent and/or the subject being treated. In some embodiments of the uses described herein, the reduction of accumulation of said agent in the kidney is measured about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours after treatment of the subject with the agent. Preferably, the reduction of accumulation of said agent in the kidney is measured about 2 hours, about 4 hours, or about 6 hours after treatment of the subject with the agent. In some embodiments of the uses described herein, the reduction of accumulation of said agent in the kidney is measured between about 1 hour and about 96 hours after treatment of the subject with the agent, between about 1 hour and about 72 hours after treatment of the subject with the agent, between about 1 hour and about 48 hours after treatment of the subject with the agent, between about 1 hour and about 24 hours after treatment of the subject with the agent, between about 1 hour and about 18 hours after treatment of the subject with the agent, between about 1 hour and about 12 hours after treatment of the subject with the agent, between about 1 hour and about 6 hours after treatment of the subject with the agent, or between about 2 hour and about 6 hours after treatment of the subject with the agent.

Any device or method known in the art for detecting radioactive emissions of drug moieties such as radionuclides in a subject is suitable to measure and quantify the reduction in kidney accumulation described herein. For example, methods such as single photon emission computerized tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from a radiolabeled conjugate described herein. Positron emission tomography (PET) is another suitable technique for detecting radiation in a subject. Furthermore, nuclear magnetic resonance (NMR)-based methods (e.g, magnetic resonance spectroscopy (MRS) and magnetic resonance imaging (MRI)) or any other imaging technique known to one of skill in the art (including, but not limited to, computed tomography (CT)) may be combined with methods that are suitable for detecting the radioactive emissions of radionuclides. In cases where the agent does not comprise a radioactivity emitting drug moiety, such as when the agent comprises a drug moiety such as a cytotoxin (therapeutic) or a fluorophore (diagnostic), any device or method known in the art for detecting said drug moiety in a subject is suitable to measure and quantify the reduction in kidney accumulation described herein.

Uses described herein are generally performed on a subject in need of a therapy or diagnosis and treated for this purpose with said agent. Such subject can be a subject having, diagnosed with, suspected of having, or at risk for developing a disease such as cancer. A determination of the need for treatment will typically be assessed by a history and physical exam consistent with the disease or condition at issue.

The administration of the agent performed in the uses described herein may be any suitable systemic administration. Such systemic administration is preferably a parenteral administration, and includes for instance intravenous (i.v.), subcutaneous, intramuscular or intradermal administration.

Accordingly, in one embodiment, said subject is treated with said agent by systemic administration of said agent. In a more particular embodiment, said systemic administration of said agent is by parenteral administration. In a preferred embodiment, said administration of said agent is by intravenous administration.

The administration of the designed ankyrin repeat domain for use described herein may be any suitable systemic administration. Such systemic administration is preferably a parenteral administration, and includes for instance intravenous (i.v.), subcutaneous, intramuscular or intradermal administration.

Accordingly, in one embodiment, the administration of the designed ankyrin repeat domain for use is a systemic administration. In a more particular embodiment, the administration of the designed ankyrin repeat domain for use is a parenteral administration. In a preferred embodiment, the administration of the designed ankyrin repeat domain for use is an intravenous administration.

In preferred embodiments, the designed ankyrin repeat domain for use is administered concomitantly with the agent. In preferred embodiments, the designed ankyrin repeat domain for use is co-administered with the agent. In further embodiments, the designed ankyrin repeat domain for use is administered sequentially with the agent.

In some embodiments, the designed ankyrin repeat domain for use is administered to the subject at a molar ratio of repeat domain to agent of about 1:1 or higher, about 5:1 or higher, about 20:1 or higher, about 30:1 or higher, about 40:1 or higher, about 50:1 or higher, about 60:1 or higher, about 70:1 or higher, about 80:1 or higher, about 90:1 or higher, about 100:1 or higher, about 200:1 or higher, about 300:1 or higher, about 400:1 or higher, about 500:1 or higher, about 600:1 or higher, about 700:1 or higher, about 800:1 or higher, about 900:1 or higher, about 1000:1 or higher, about 1100:1 or higher, about 1200:1 or higher, about 1300:1 or higher, about 1400:1 or higher, about 1500:1 or higher, about 1600:1 or higher, about 1700:1 or higher, about 1800:1 or higher, about 1900:1 or higher, about 2000:1 or higher, about 2500:1 or higher, about 3000:1 or higher, about 3500:1 or higher, about 4000:1 or higher, about 4500:1 or higher, about 5000:1 or higher, about 5500:1 or higher, or about 6000 or higher. Preferably, said repeat domain for use is administered to the subject at a molar ratio of repeat domain to agent of about 1:1 or higher, about 50:1 or higher, about 100:1 or higher, about 500:1 or higher, about 1000:1 or higher, about 1500:1 or higher, about 2000:1 or higher, about 2500:1 or higher, about 3000:1 or higher, or about 3500:1 or higher, about 4000:1 or higher, about 4500:1 or higher, about 5000:1 or higher, about 5500:1 or higher, or about 6000:1 or higher. Most preferably, said repeat domain is administered to the subject at a molar ratio of repeat domain to agent of about 50:1 or higher.

In particular embodiments in which the agent comprises a radioactive drug moiety such as a radionuclide, the effective molar ratio of repeat domain to radiolabeled agent may vary depending on the radiolabeling efficiency reached during production of that agent. Accordingly, the above molar ratios for such cases refer to molar ratios without taking into consideration any potential loss in radiolabeled agent, for instance where a percentage of radiolabeled agent being part of an amount to be administered would effectively be non-labeled (due to sub-optimal radiolabeling efficiency during the production of the radiolabeled agent). Such considerations are known to a skilled person in the art. Similar considerations apply for agents comprising a non-radioactive drug moiety as defined herein.

In some embodiments of the uses described herein, the accumulation of said agent in the kidney is evaluated in comparison to an appropriate control treatment. Such a control treatment may be an administration to a subject of said agent as control without administration of the designed ankyrin repeat domain. Accordingly, in some embodiments of the uses described herein, the accumulation of said agent in the kidney is reduced by at least 10%, at least 20%, at least 30%, at least 40% or at least 50% as compared to the accumulation of said agent in the kidney of a subject treated with said agent as a control without administration of the designed ankyrin repeat domain.

In preferred embodiments, said reduction of accumulation of the agent in the kidney is calculated as in Example 1 to 4.

In some embodiments, the designed ankyrin repeat domain for use is a non-binding repeat domain. In some embodiments, the designed ankyrin repeat domain for use does not specifically bind to a target with a dissociation constant (K_{D}) of 10⁻⁵ M or below, of 10⁻⁶ M or below, or of 10⁻⁷ M or below, preferably of 10⁻⁷ M or below.

In some embodiments, the designed ankyrin repeat domain for use has an isoelectric point (pI) in a range between about pH 4.5 and about pH 7.0, between about pH 4.5 and about pH 6.0, between about pH 4.5 and about pH 5.5, between about pH 4.5 and about pH 5.0, between about pH 4.6 and about pH 7.0, between about pH 4.6 and about pH 6.0, between about pH 4.6 and about pH 5.5, between about pH 4.6 and about pH 5.0, between about pH 4.7 and about pH 7.0, between about pH 4.7 and about pH 6.0, between about pH 4.7 and about pH 5.5, between about pH 4.5 and about pH 5.0, between about pH 4.8 and about pH 7.0, between about pH 4.8 and about pH 6.0, between about pH 4.8 and about pH 5.5, between about pH 4.8 and about pH 5.0.

In some embodiments, the designed ankyrin repeat domain for use has an isoelectric point (pI) in a range between about pH 4.5 and about pH 6.5, preferably between about pH 4.6 and about pH 6.0, and more preferably between about pH 4.7 and about pH 5.5.

In some embodiments, the designed ankyrin repeat domain for use has an isoelectric point (pI) in a range between about pH 4.5 and about pH 9.4, between about pH 4.5 and about pH 9.0, between about pH 4.5 and about pH 8.5, between about pH 4.5 and about pH 8.0, between about pH 4.5 and about pH 7.5, between about pH 4.6 and about pH 9.4, between about pH 4.6 and about pH 9.0, between about pH 4.6 and about pH 8.5, between about pH 4.6 and about pH 8.0, between about pH 4.6 and about pH 7.5, between about pH 4.7 and about pH 9.4, between about pH 4.7 and about pH 9.0, between about pH 4.7 and about pH 8.5, between about pH 4.5 and about pH 8.0, between about pH 4.7 and about pH 7.5, between about pH 4.8 and about pH 9.4, between about pH 4.8 and about pH 9.0, between about pH 4.8 and about pH 8.5, between about pH 4.8 and about pH 8.0, and between about pH 4.8 and about pH 7.5. In some embodiments, the designed ankyrin repeat domain for use comprises an N-terminal capping module, a C-terminal capping module and one or more internal repeat module(s).

In some embodiments, the designed ankyrin repeat domains used in the uses described herein may be obtained by substitution of an amino acid, methods to perform such substitutions are well known in the art and include mutagenesis of the cDNA encoding the described repeat domains.

Residues selected for substitutions can be located at randomized or non-randomized positions of the repeat domain. Accordingly, in some embodiments, the substituted residues are selected among residues located at non-randomized positions of said repeat domain. In other embodiments the substituted residues are selected among residues located at randomized positions of said repeat domain. In some embodiments the substituted residues are selected among residues located at randomized and non-randomized positions of said repeat domain.

In other embodiments the substituted residues are selected among all residues comprised in said repeat domain. All residues in this sense shall mean any of the residues located at a randomized or non-randomized position comprised in a designed ankyrin repeat domain described herein. Preferred randomized positions are shown in Table A. Table B shows preferred non-randomized positions of the designed ankyrin repeat domains described herein.

TABLE A

| Repeat modules | Representative sequence | Randomized positions |
|---|---|---|
| N-terminal capping | SEQ ID NO: 5 | 4, 8, 11 and 12 |
| module | | |
| C-terminal capping | SEQ ID NO: 6 | 3, 4, 6, 14 and 15 |
| module | | |
| Internal repeat | SEQ ID NO: 7 | 3, 4, 6, 11, 14 and 15 |
| module | | |

TABLE B

| | Representative | |
|---|---|---|
| Repeat modules | sequence | Non-randomized positions |
| N-terminal capping | SEQ ID NO: 5 | 1 to 3, 5 to 7, |
| module | | 9, 10, 13 to 30 |
| C-terminal capping | SEQ ID NO: 6 | 1, 2, 5, 7 to 13, 16 to 28 |
| module | | |
| Internal repeat | SEQ ID NO: 7 | 1, 2, 5, 7 to 10, |
| module | | 12, 13, 16 to 33 |

In some embodiments of the uses described herein, the substitutions are only performed in the N-terminal capping module. In some embodiments of the uses described herein, the substitutions are only performed in the C-terminal capping module. In some embodiments of the uses described herein, the substitutions are only performed in the C-terminal and in the in the N-terminal capping modules. In some embodiments of the uses described herein, the substitutions are only performed in the internal repeat module(s).

Examples of conservative and other exemplary amino acid residue substitutions that may occur in designed ankyrin repeat domains and proteins described herein are shown in Table C. In preferred embodiments, the substitute amino acid is not cysteine, glycine, or proline.

TABLE C

| | Conservative | |
|---|---|---|
| Original Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; Norleucine |

In some embodiments, the designed ankyrin repeat domain for use comprises:
(a) an N-terminal capping module having the amino acid sequence of SEQ ID NO: 5 or any variant thereof having at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with SEQ ID NO: 5, and/or
(b) a C-terminal capping module having the amino acid sequence of SEQ ID NO: 6 or any variant thereof having at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with SEQ ID NO: 6, and/or
(c) one or more internal repeat module(s) each independently having the amino acid sequence of SEQ ID NO: 7 or any variant thereof having at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with SEQ ID NO: 7.

Accordingly, in one embodiment, the designed ankyrin repeat domain for use comprises:
(a) an N-terminal capping module having the amino acid sequence of SEQ ID NO: 5 or any variant thereof having at least 90% sequence identity with SEQ ID NO: 5, and/or
(b) a C-terminal capping module having the amino acid sequence of SEQ ID NO: 6 or any variant thereof having at least 90% sequence identity with SEQ ID NO: 6, and/or
(c) one or more internal repeat module(s) each independently having the amino acid sequence of SEQ ID NO: 7 or any variant thereof having at least 90% sequence identity with SEQ ID NO: 7.

In some embodiments, the designed ankyrin repeat domain for use comprises:
(a) an N-terminal capping module having the amino acid sequence of SEQ ID NO: 5 or any variant of SEQ ID NO: 5 wherein:
   (i) up to 1, up to 2, up to 3 or up to 4 amino acids selected from the randomized positions corresponding to positions 4, 8, 11 and 12 of SEQ ID NO: 5 are substituted by a different amino acid, and
   (ii) up to 1, up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25 or up to 26 amino acids selected from the non-randomized positions corresponding to positions 1, 2, 3, 5, 6, 7, 9, 10, 13, 14, 15, 16, 17, 18, 19, 20, 21, 22, 23, 24, 25, 26, 27, 28, 29 and 30 of SEQ ID NO: 5 are substituted by a different amino acid, and/or
(b) a C-terminal capping module having the amino acid sequence of SEQ ID NO: 6 or any variant of SEQ ID NO: 6 wherein:
   (i) up to 1, up to 2, up to 3, up to 4 or up to 5 amino acids selected from the randomized positions corresponding to positions 3, 4, 6, 14 and 15 of SEQ ID NO: 6 are substituted by a different amino acid, and
   (ii) up to 1, up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22 or up to 23 amino acids selected from the non-randomized positions corresponding to positions 1, 2, 5, 7, 8, 9, 10, 11, 12, 13, 16, 17, 18, 19, 20, 21, 22, 23, 24, 25, 26, 27 and 28 of SEQ ID NO: 6 are substituted by a different amino acid, and/or
(c) one or more internal repeat module(s) each independently having the amino acid sequence of SEQ ID NO: 7 or any variant of SEQ ID NO: 7 wherein:
   (i) up to 1, up to 2, up to 3, up to 4, up to 5 or up to 6 amino acids selected from the randomized positions corresponding to positions 3, 4, 6, 11 14 and 15 of SEQ ID NO: 7 are substituted by a different amino acid, and
   (ii) up to 1, up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26 or up to 27 amino acids selected from the non-randomized positions corresponding to positions 1, 2, 5, 7, 8, 9, 10, 12, 13, 16, 17, 18, 19, 20, 21, 22, 23, 24, 25, 26, 27, 28, 29, 30, 31, 32 and 33 of SEQ ID NO: 7 are substituted by a different amino acid.

Accordingly, in one embodiment, the designed ankyrin repeat domain for use comprises:
(a) an N-terminal capping module having the amino acid sequence of SEQ ID NO: 5 or any variant of SEQ ID NO: 5 wherein:
   (i) up to 4 amino acids selected from the randomized positions corresponding to positions 4, 8, 11 and 12 of SEQ ID NO: 5 are substituted by a different amino acid, and
   (ii) up to 15 amino acids selected from the non-randomized positions corresponding to positions 1, 2, 3, 5, 6, 7, 9, 10, 13, 14, 15, 16, 17, 18, 19, 20, 21, 22, 23, 24, 25, 26, 27, 28, 29 and 30 of SEQ ID NO: 5 are substituted by a different amino acid, and/or
(b) a C-terminal capping module having the amino acid sequence of SEQ ID NO: 6 or any variant of SEQ ID NO: 6 wherein:
   (i) up to 5 amino acids selected from the randomized positions corresponding to positions 3, 4, 6, 14 and 15 of SEQ ID NO: 6 are substituted by a different amino acid, and
   (ii) up to 15 amino acids selected from the non-randomized positions corresponding to positions 1, 2, 5, 7, 8, 9, 10, 11, 12, 13, 16, 17, 18, 19, 20, 21, 22, 23, 24, 25, 26, 27 and 28 of SEQ ID NO: 6 are substituted by a different amino acid, and/or
(c) one or more internal repeat module(s) each independently having the amino acid sequence of SEQ ID NO: 7 or any variant of SEQ ID NO: 7 wherein:
   (i) up to 6 amino acids selected from the randomized positions corresponding to positions 3, 4, 6, 11 14 and 15 of SEQ ID NO: 7 are substituted by a different amino acid, and
   (ii) up to 15 amino acids selected from the non-randomized positions corresponding to positions 1, 2, 5, 7, 8, 9, 10, 12, 13, 16, 17, 18, 19, 20, 21, 22, 23, 24, 25, 26, 27, 28, 29, 30, 31, 32 and 33 of SEQ ID NO: 7 are substituted by a different amino acid.

In some embodiments, the designed ankyrin repeat domain for use comprises two or more internal repeat modules. In more particular embodiments, said internal repeat modules comprised in the designed ankyrin repeat domain for use have at least 80%, at least 85%, at least 90%, at least 91%, at least 92%, at least 93%, at least 94%, at least 95%, at least 96%, at least 97%, at least 98%, at least 99% or 100% sequence identity between each other. In some embodiments, the residues located at randomized positions of the internal repeat modules comprised in the ankyrin repeat domain for use are identical between each of said internal repeat modules. In preferred embodiments, said randomized positions correspond to positions 3, 4, 6, 11, 14 and 15 of the internal repeat module, numbered relative to SEQ ID NO: 7.

In some embodiments, the designed ankyrin repeat domain for use comprises one, two, three, four, five, six, seven, eight or nine internal repeat modules. In preferred embodiments, the designed ankyrin repeat domain for use comprises two internal repeat modules. In some embodiments, the designed ankyrin repeat domain for use comprises exactly one, two, three, four, five, six, seven, eight or nine internal repeat modules.

In some embodiments, the designed ankyrin repeat domain for use comprises an amino acid sequence selected from the group consisting of (1) SEQ ID NO: 1 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 1. Thus, in a more particular embodiment, said designed repeat domain for use comprises an amino acid sequence selected from the group consisting of (1) SEQ ID NO: 1 and (2) sequences with at least 80% amino acid sequence identity with SEQ ID NO: 1.

In some embodiments, the designed ankyrin repeat domain for use comprises an amino acid sequence selected from the group consisting of (1) SEQ ID NO: 11 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 11. Thus, in a more particular embodiment, said designed repeat domain for use comprises an amino acid sequence selected from the group consisting of (1) SEQ ID NO: 11 and (2) sequences with at least 80% amino acid sequence identity with SEQ ID NO: 11.

In some embodiments, the designed ankyrin repeat domain for use comprises an amino acid sequence selected from the group consisting of (1) SEQ ID NO: 12 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 12. Thus, in a more particular embodiment, said designed repeat domain for use comprises an amino acid sequence selected from the group consisting of (1) SEQ ID NO: 12 and (2) sequences with at least 80% amino acid sequence identity with SEQ ID NO: 12.

In some embodiments, the designed ankyrin repeat domain for use comprises an amino acid sequence selected from the group consisting of (1) SEQ ID NO: 13 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 13. Thus, in a more particular embodiment, said designed repeat domain for use comprises an amino acid sequence selected from the group consisting of (1) SEQ ID NO: 13 and (2) sequences with at least 80% amino acid sequence identity with SEQ ID NO: 13.

In some embodiments, the designed ankyrin repeat domain for use comprises an amino acid sequence selected from the group consisting of (1) SEQ ID NO: 14 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 14. Thus, in a more particular embodiment, said designed repeat domain for use comprises an amino acid sequence selected from the group consisting of (1) SEQ ID NO: 14 and (2) sequences with at least 80% amino acid sequence identity with SEQ ID NO: 14.

In some embodiments, the designed ankyrin repeat domain for use comprises an amino acid sequence selected from the group consisting of (1) SEQ ID NO: 15 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 15. Thus, in a more particular embodiment, said designed repeat domain for use comprises an amino acid sequence selected from the group consisting of (1) SEQ ID NO: 15 and (2) sequences with at least 80% amino acid sequence identity with SEQ ID NO: 15.

In some embodiments, the designed ankyrin repeat domain for use comprises an amino acid sequence selected from the group consisting of (1) SEQ ID NO: 16 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 16. Thus, in a more particular embodiment, said designed repeat domain for use comprises an amino acid sequence selected from the group consisting of (1) SEQ ID NO: 16 and (2) sequences with at least 80% amino acid sequence identity with SEQ ID NO: 16.

In some embodiments, the designed ankyrin repeat domain for use comprises an amino acid sequence selected from the group consisting of (1) SEQ ID NO: 17 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 17. Thus, in a more particular embodiment, said designed repeat domain for use comprises an amino acid sequence selected from the group consisting of (1) SEQ ID NO: 17 and (2) sequences with at least 80% amino acid sequence identity with SEQ ID NO: 17.

In some embodiments, the designed ankyrin repeat domain for use is administered to the subject in form of a pharmaceutical composition comprising said repeat domain and optionally at least one pharmaceutically acceptable carrier or diluent. In some embodiments, said pharmaceutical composition also comprises the therapeutic and/or diagnostic agent.

Such pharmaceutical compositions may be prepared using methods known in the art, and are further described below.

Furthermore, the sequence of any repeat domain disclosed herein may optionally comprise at its N-terminus, a G, an S, or a GS. Furthermore, the sequence of any repeat domain disclosed herein may optionally have A at the second last position substituted with L and/or A at the last position substituted with N.

### Recombinant Proteins

In another aspect, the invention provides a recombinant protein for use in reducing accumulation of a therapeutic and/or diagnostic agent in the kidney of a subject treated with said agent, wherein the recombinant protein is administered to the subject in an amount effective to reduce accumulation of said therapeutic and/or diagnostic agent in the kidney, and wherein said recombinant protein comprises the designed ankyrin repeat domain for use according to the invention.

In some embodiments, said recombinant protein for use has a molecular weight of 69 kDa or less, 65 kDa or less, 60 kDa or less, 55 kDa or less, 50 kDa or less, 45 kDa or less, 40 kDa or less, 35 kDa or less, or 30 kDa or less.

In some embodiments, said recombinant protein for use is administered to the subject in form of a pharmaceutical composition comprising said recombinant protein and optionally at least one pharmaceutically acceptable carrier or diluent. In some embodiments, said pharmaceutical composition also comprises the therapeutic and/or diagnostic agent.

The designed ankyrin repeat domains of the invention can be genetically fused to further components, such as, e.g., a drug moiety, a protein or an agent, and such fusions are also referred to as “recombinant protein”. Linkers known in the art may be used between repeat domains in such repeat proteins (see, e.g., WO 2021/116469) or between a repeat domain and said further component. Such recombinant proteins are in particular envisioned for use in medicine, more particularly for use in methods of reducing accumulation of a therapeutic and/or diagnostic agent in the kidney of a subject treated with said agent, as further described below.

In some embodiments, the recombinant proteins for use according to the invention comprise one or more additional designed ankyrin repeat domains.

Embodiments and considerations relating to the therapeutic and/or diagnostic agent, the administration, the subject and the pharmaceutical composition described herein for the designed ankyrin repeat domain for use similarly apply to the above recombinant protein for use.

### Nucleic Acids, Vectors and Host Cells

In another aspect, the invention relates to an isolated nucleic acid encoding the amino acid sequence of the designed ankyrin repeat domain described herein or of the recombinant protein described herein. Accordingly, in one embodiment, the invention relates to an isolated nucleic acid encoding the designed ankyrin repeat domain for use according to the invention, or the recombinant protein for use according to the invention. In one embodiment, the invention relates to an isolated nucleic acid encoding the amino acid sequence of the recombinant protein for use according to the present invention. In one embodiment, the invention relates to an isolated nucleic acid encoding the amino acid sequence of the designed ankyrin repeat domain for use according to the present invention.

Furthermore, the invention relates to vectors comprising any nucleic acid of the invention. Accordingly, in another aspect, the invention provides a recombinant expression vector comprising a nucleic acid according to the invention, wherein the vector optionally comprises an expression control sequence, allowing expression in prokaryotic or eukaryotic host cells of the encoded polypeptide, operably linked to said nucleic acid. The nucleic acid sequence can be inserted in the recombinant vector by methods well known to a person skilled in the art such as, for example, those that are described in MOLECULAR CLONING: A LABORATORY MANUAL, Sambrook et al, 4th Ed., Cold Spring Harbor Laboratory Press, Cold Spring Harbor, N Y., 2001.

Nucleic acids are well known to the skilled person in the art. Nucleic acids were used to produce designed ankyrin repeat domains or recombinant binding proteins of the invention in E. coli, e.g. as further described in Example 1 or in U.S. Pat. No. 7,417,130.

In another aspect, the invention provides a host cell comprising a recombinant expression vector according to the invention. The host cell can be, for example, bacterial cells such as Escherichia coli or Streptomyces, fungal cells such as Aspergillus and yeasts such as Saccharomyces, insect cells, mammalian cells such as Chinese Hamster Ovary (CHO) cells, CI 27 mouse cell line, BHK cell line of Syrian hamster cells, Human Embryonic Kidney 293 (HEK 293) cells. In some embodiment, the host cell is a CHO cell or a HEK 293 cell. The host cells can be used, for example, to express a recombinant protein of the invention.

### Compositions

The invention further relates to pharmaceutical compositions comprising one or more of a designed ankyrin repeat domain, a recombinant protein, a nucleic acid and/or a recombinant expression vector described herein and a pharmaceutically acceptable carrier or diluent. Accordingly, in one embodiment, the invention relates to pharmaceutical compositions comprising one or more of a designed ankyrin repeat domain for use according to the invention, a recombinant protein for use according to the invention, a nucleic acid and/or a recombinant expression vector according to the invention and a pharmaceutically acceptable carrier or diluent The invention also relates to uses and methods of reducing accumulation of a therapeutic and/or diagnostic agent in the kidney of subject treated with said agent using the pharmaceutical compositions disclosed herein.

The pharmaceutical compositions described herein may be prepared using methods known in the art.

The pharmaceutical compositions optionally comprise a pharmaceutically acceptable carrier or excipient or diluent. Standard pharmaceutical carriers include a phosphate buffered saline solution, water, emulsions such as an oil/water or water/oil emulsion, and various types of wetting agents.

The pharmaceutical compositions of the invention may comprise any other pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, colouring agents, desiccants, detergents, diluents, disinfectants, disintegrants, dispersing agents, dissolution enhancing agents, dyes, emollients, emulsifying agents, emulsion stabilizers, fillers, film forming agents, flavour enhancers, flavouring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Macadk Publishing Co., Easton, Pa., 1980), which is incorporated by reference in its entirety.

In one embodiment, the invention provides a pharmaceutical composition comprising one or more of: (i) a designed ankyrin repeat domain for use according to the invention, (ii) a recombinant protein for use according to the invention, (iii) a nucleic acid according to the invention, and/or (iv) a recombinant expression vector according to the invention, and optionally at least one pharmaceutically acceptable carrier or diluent.

### Uses and Methods of the Invention

In another aspect, the invention provides a method of reducing accumulation of a therapeutic and/or diagnostic agent in the kidney of a subject treated with said agent, the method comprising the step of administering to said subject an effective amount of a designed ankyrin repeat domain or of a recombinant protein comprising a designed ankyrin repeat domain.

In some embodiments, the invention provides a method for reducing accumulation of a therapeutic and/or diagnostic agent in the kidney of a subject treated with said agent, the method comprising administering to the subject a designed ankyrin repeat domain or a recombinant protein comprising a designed ankyrin repeat domain in an amount effective to reduce accumulation of said therapeutic and/or diagnostic agent in the kidney.

Further provided is a designed repeat domain or a recombinant protein comprising a designed ankyrin repeat domain for use in a method of reducing accumulation of a therapeutic and/or diagnostic agent in the kidney of a subject treated with said agent, the method comprising the step of administering to said subject an effective amount of the designed ankyrin repeat domain or the recombinant protein to reduce accumulation of said agent in the kidney.

In one embodiment, the invention relates to the use of a designed repeat domain or a recombinant protein comprising a designed ankyrin repeat domain for reducing accumulation of a therapeutic and/or diagnostic agent in the kidney of a subject being treated with said agent, wherein the designed repeat domain or the recombinant protein is administered to said subject in an effective amount to reduce accumulation of said therapeutic and/or diagnostic agent in the kidney.

In one embodiment, the invention relates to the use of a designed ankyrin repeat domain or a recombinant protein comprising a designed ankyrin repeat domain, for manufacturing of a medicament.

In one embodiment, the invention relates to the use of the designed ankyrin repeat domain or a recombinant protein comprising a designed ankyrin repeat domain, for manufacturing of a medicament for reducing accumulation of a therapeutic and/or diagnostic agent in the kidney of a subject being treated with said agent.

In a further embodiment, the invention relates to the use of the designed ankyrin repeat domain or a recombinant protein comprising a designed ankyrin repeat domain for the manufacture of a medicament that is used for reducing accumulation of a therapeutic and/or diagnostic agent in the kidney of a subject being treated with said agent.

The therapeutic and/or diagnostic agents encompassed in the uses and methods of the invention include any agent that has a desired therapeutic and/or diagnostic property and that can be administered to a subject in need of a therapy and/or a diagnosis. Therapeutic and/or diagnostic agents used in the field of nuclear medicine such as in radiopharmaceutical therapy or diagnosis of cancer, and agents such as cytotoxic-conjugated proteins for cancer therapy are preferred. A radiopharmaceutical generally refers to any radioactive compound that can be used as a therapeutic and/or diagnostic agent. Radiopharmaceuticals may comprise a radionuclide and a binding moiety so that the radionuclide is delivered to a target site, e.g. the tumor tissue, in a targeted manner. Radiopharmaceutical agents having both therapeutic and diagnostic properties may also be referred to as theranostic agents.

Accordingly, in some embodiments of the uses and methods described herein, said therapeutic and/or diagnostic agent comprises a binding moiety. In some embodiments, said binding moiety comprises a small organic molecule, a peptide, a monoclonal antibody fragment, or an alternative scaffold as further described herein. In some embodiments, said binding moiety binds to a target with a dissociation constant (K_{D}) of about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less.

Alternative scaffolds include any polypeptides or proteins comprising a binding domain that is capable of binding a target and that is not derived from an antibody or immunoglobulin molecule. The binding domain of alternative scaffolds may comprise or may be derived from a variety of different polypeptide or protein structures. Alternative scaffolds include, but are not limited to, adnectins (monobodies), affibodies, affilins, affimers and aptamers, affitins, alphabodies, anticalins, armadillo repeat protein-based scaffolds, atrimers, avimers, ankyrin repeat protein-based scaffolds (such as DARPin proteins), fynomers, knottins, and Kunitz domain peptides. Such alternative scaffolds are described further above.

In preferred embodiments of the uses and methods described herein, said binding moiety comprises a designed ankyrin repeat domain with binding specificity for a target. In other embodiments, said binding moiety consists of a designed ankyrin repeat domain with binding specificity for a target.

In some embodiments of the uses and methods described herein, said therapeutic and/or diagnostic agent comprises a drug moiety and/or a binding moiety. Accordingly, in some embodiments of the uses and methods described herein, said therapeutic and/or diagnostic agent comprises a drug moiety. In some embodiments of the uses and methods described herein, said therapeutic and/or diagnostic agent comprises a binding moiety.

Preferably, in some embodiments of the uses and methods described herein, said therapeutic and/or diagnostic agent comprises a drug moiety and a binding moiety. In particular embodiments, said drug moiety is covalently or non-covalently linked to the binding moiety. In other particular embodiments of the uses and methods described herein, said drug moiety is linked to said binding moiety by a chelator. Appropriate chelators may be selected depending on the nature of the binding and drug moieties. In a particular embodiment of the uses is s and methods described herein, said chelator diethylenetriaminepentaacetic acid (DTPA).

In some embodiments of the uses and methods described herein, said drug moiety is a therapeutic and/or diagnostic moiety. In preferred embodiments of the uses and methods described herein, said drug moiety is a radionuclide. The choice of said drug moiety may depend on the intended purpose of the of the agent (e.g. diagnostic vs. therapeutic).

In particular embodiments of the uses and methods described herein, said drug moiety is a therapeutic moiety. In some embodiments of the uses and methods described herein, said therapeutic moiety is a toxin. In more particular embodiments of the uses and methods described herein, said therapeutic moiety is a radionuclide as defined herein. In other more particular embodiments of the uses and methods described herein, said therapeutic moiety is a cytotoxin as defined herein.

In particular embodiments of the uses and methods described herein, said drug moiety is a diagnostic moiety. In a more particular embodiment of the uses and methods described herein, said diagnostic moiety is a fluorophore, a chromophore, an imaging agent or a radionuclide.

Accordingly, in a preferred embodiment of the uses and methods described herein, said therapeutic and/or diagnostic agent comprises a binding moiety and a drug moiety, wherein said binding moiety comprises or consists of a designed ankyrin repeat domain with binding specificity for a target. In particular embodiments of the uses and methods described herein, said designed ankyrin repeat domain is covalently or non-covalently linked to said drug moiety. In particular embodiments of the uses and methods described herein, said designed ankyrin repeat domain is linked to said drug moiety by a chelator. In particular embodiments of the uses and methods described herein, said chelator is diethylenetriaminepentaacetic acid (DTPA). In particular embodiments of the uses and methods described herein, said drug moiety is a radionuclide. In some particular embodiments of the uses and methods described herein, said radionuclide is indium-111. In other particular embodiments of the uses and methods described herein, said drug moiety is a cytotoxin.

In further embodiments of the uses and methods described herein, said ankyrin repeat domain comprised in said binding moiety binds specifically to a target. In some embodiments, said ankyrin repeat domain comprised in said binding moiety binds to said target with a dissociation constant (K_{D}) of about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less.

In some preferred embodiments, the amino acid sequence of said designed ankyrin repeat domain or recombinant protein is different from the amino acid sequence of the designed ankyrin repeat domain comprised in said binding moiety. In some embodiments, the amino acid sequence of said designed ankyrin repeat domain or recombinant protein differs in sequence identity from the amino acid sequence comprised in said binding moiety by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or by 100%, In some preferred embodiments, the designed ankyrin repeat domain or recombinant protein is not comprised in said binding moiety.

The timepoint at which the accumulation of said agent in the kidney is determined may depend on the chemical properties of the agent and/or the subject being treated. In some embodiments of the uses and methods described herein, the reduction of accumulation of said agent in the kidney is measured about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours after treatment of the subject with the agent. Preferably, the reduction of accumulation of said agent in the kidney is measured about 2 hours, about 4 hours, or about 6 hours after treatment of the subject with the agent. In some embodiments of the uses and methods described herein, the reduction of accumulation of said agent in the kidney is measured between about 1 hour and about 96 hours after treatment of the subject with the agent, between about 1 hour and about 72 hours after treatment of the subject with the agent, between about 1 hour and about 48 hours after treatment of the subject with the agent, between about 1 hour and about 24 hours after treatment of the subject with the agent, between about 1 hour and about 18 hours after treatment of the subject with the agent, between about 1 hour and about 12 hours after treatment of the subject with the agent, between about 1 hour and about 6 hours after treatment of the subject with the agent, or between about 2 hour and about 6 hours after treatment of the subject with the agent.

Any device or method known in the art for detecting radioactive emissions of drug moieties such as radionuclides in a subject is suitable to measure and quantify the reduction in kidney accumulation described herein. For example, methods such as single photon emission computerized tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from a radiolabeled conjugate described herein. Positron emission tomography (PET) is another suitable technique for detecting radiation in a subject. Furthermore, nuclear magnetic resonance (NMR)-based methods (e.g, magnetic resonance spectroscopy (MRS) and magnetic resonance imaging (MRI)) or any other imaging technique known to one of skill in the art (including, but not limited to, computed tomography (CT)) may be combined with methods that are suitable for detecting the radioactive emissions of radionuclides. In cases where the agent does not comprise a radioactivity emitting drug moiety, such as when the agent comprises a drug moiety such as a cytotoxin (therapeutic) or a fluorophore (diagnostic), any device or method known in the art for detecting said drug moiety in a subject is suitable to measure and quantify the reduction in kidney accumulation described herein.

Uses and methods described herein are generally performed on a subject in need of a therapy or diagnosis and treated for this purpose with said agent. Such subject can be a subject having, diagnosed with, suspected of having, or at risk for developing a disease such as cancer. A determination of the need for treatment will typically be assessed by a history and physical exam consistent with the disease or condition at issue.

The administration of the agent performed in the uses and methods described herein may be any suitable systemic administration. Such systemic administration is preferably a parenteral administration, and includes for instance intravenous (i.v.), subcutaneous, intramuscular or intradermal administration.

Accordingly, in one embodiment, said subject is treated with said agent by systemic administration of said agent. In a more particular embodiment, said systemic administration of said agent is by parenteral administration. In a preferred embodiment, said administration of said agent is by intravenous administration.

The administration of said designed ankyrin repeat domain or recombinant protein comprising a designed ankyrin repeat protein may be any suitable systemic administration. Such systemic administration is preferably a parenteral administration, and includes for instance intravenous (i.v.), subcutaneous, intramuscular or intradermal administration.

Accordingly, in one embodiment, the administration of said designed ankyrin repeat domain or recombinant protein is a systemic administration. In a more particular embodiment, the administration of said designed ankyrin repeat domain or recombinant protein is a parenteral administration. In a preferred embodiment, the administration of said designed ankyrin repeat domain or recombinant protein is an intravenous administration.

In preferred embodiments, said designed ankyrin repeat domain or recombinant protein is administered concomitantly with the agent. In preferred embodiments, the designed ankyrin repeat domain or recombinant protein is co-administered with the agent. In further embodiments, the designed ankyrin repeat domain or recombinant protein is administered sequentially with the agent.

In some embodiments, the designed ankyrin repeat domain or recombinant protein is administered to the subject at a molar ratio of designed ankyrin repeat domain to agent or recombinant protein to agent of about 1:1 or higher, about 5:1 or higher, about 20:1 or higher, about 30:1 or higher, about 40:1 or higher, about 50:1 or higher, about 60:1 or higher, about 70:1 or higher, about 80:1 or higher, about 90:1 or higher, about 100:1 or higher, about 200:1 or higher, about 300:1 or higher, about 400:1 or higher, about 500:1 or higher, about 600:1 or higher, about 700:1 or higher, about 800:1 or higher, about 900:1 or higher, about 1000:1 or higher, about 1100:1 or higher, about 1200:1 or higher, about 1300:1 or higher, about 1400:1 or higher, about 1500:1 or higher, about 1600:1 or higher, about 1700:1 or higher, about 1800:1 or higher, about 1900:1 or higher, about 2000:1 or higher, about 2500:1 or higher, about 3000:1 or higher, about 3500:1 or higher, about 4000:1 or higher, about 4500:1 or higher, about 5000:1 or higher, about 5500:1 or higher, or about 6000 or higher. Preferably, the designed ankyrin repeat domain or recombinant protein is administered to the subject at a molar ratio of repeat domain to agent or recombinant protein to agent of about 1:1 or higher, about 50:1 or higher, about 100:1 or higher, about 500:1 or higher, about 1000:1 or higher, about 1500:1 or higher, about 2000:1 or higher, about 2500:1 or higher, about 3000:1 or higher, or about 3500:1 or higher, about 4000:1 or higher, about 4500:1 or higher, about 5000:1 or higher, about 5500:1 or higher, or about 6000:1 or higher. Most preferably, the designed ankyrin repeat domain or recombinant protein is administered to the subject at a molar ratio of repeat domain to agent or recombinant protein to agent of about 50:1 or higher.

In particular embodiments in which the agent comprises a radioactive drug moiety such as a radionuclide, the effective molar ratio of repeat domain to radiolabeled agent may vary depending on the radiolabeling efficiency reached during production of that agent. Accordingly, the above molar ratios for such cases refer to molar ratios without taking into consideration any potential loss in radiolabeled agent, for instance where a percentage of radiolabeled agent being part of an amount to be administered would effectively be non-labeled (due to sub-optimal radiolabeling efficiency during the production of the radiolabeled agent). Such considerations are known to a skilled person in the art. Similar considerations apply for agents comprising a non-radioactive drug moiety as defined herein.

In some embodiments of the uses and methods described herein, the accumulation of said agent in the kidney is evaluated in comparison to an appropriate control treatment. Such a control treatment may be an administration to a subject of said agent as control without administration of said designed ankyrin repeat domain or recombinant protein. Accordingly, in some embodiments of the uses and methods described herein, the accumulation of said agent in the kidney is reduced by at least 10%, at least 20%, at least 30%, at least 40% or at least 50% as compared to the accumulation of said agent in the kidney of a subject treated with said agent as a control without administration of said designed ankyrin repeat domain or recombinant protein.

In preferred embodiments, said reduction of accumulation of the agent in the kidney is calculated as in Example 1 to 4.

In one embodiment, the invention provides a designed ankyrin repeat domain as disclosed herein or a recombinant protein comprising a designed ankyrin repeat domain as described herein for use as a medicament.

In some embodiments of the uses and methods described herein, said designed ankyrin repeat domain is a non-binding repeat domain. In some embodiments, the designed ankyrin repeat domain does not specifically bind to a target with a dissociation constant (K_{D}) of 10⁻⁵ M or below, of 10⁻⁶ M or below, or of 10⁻⁷ M or below, preferably of 10⁻⁷ M or below.

Accordingly, in one embodiment, the invention provides a designed ankyrin repeat domain or a recombinant protein comprising a designed ankyrin repeat domain, wherein the designed ankyrin repeat domain does not specifically bind to a target with a dissociation constant (K_{D}) of 10⁻⁷ M or below, for use as a medicament.

In some embodiments of the uses and methods described herein, said designed ankyrin repeat domain has an isoelectric point (pI) in a range between about pH 4.5 and about pH 7.0, between about pH 4.5 and about pH 6.0, between about pH 4.5 and about pH 5.5, between about pH 4.5 and about pH 5.0, between about pH 4.6 and about pH 7.0, between about pH 4.6 and about pH 6.0, between about pH 4.6 and about pH 5.5, between about pH 4.6 and about pH 5.0, between about pH 4.7 and about pH 7.0, between about pH 4.7 and about pH 6.0, between about pH 4.7 and about pH 5.5, between about pH 4.5 and about pH 5.0, between about pH 4.8 and about pH 7.0, between about pH 4.8 and about pH 6.0, between about pH 4.8 and about pH 5.5, between about pH 4.8 and about pH 5.0.

In some embodiments, said designed ankyrin repeat domain has an isoelectric point (pI) in a range between about pH 4.5 and about pH 6.5, preferably between about pH 4.6 and about pH 6.0, and more preferably between about pH 4.7 and about pH 5.5.

In some embodiments, said designed ankyrin repeat domain has an isoelectric point (pI) in a range between about pH 4.5 and about pH 9.4, between about pH 4.5 and about pH 9.0, between about pH 4.5 and about pH 8.5, between about pH 4.5 and about pH 8.0, between about pH 4.5 and about pH 7.5, between about pH 4.6 and about pH 9.4, between about pH 4.6 and about pH 9.0, between about pH 4.6 and about pH 8.5, between about pH 4.6 and about pH 8.0, between about pH 4.6 and about pH 7.5, between about pH 4.7 and about pH 9.4, between about pH 4.7 and about pH 9.0, between about pH 4.7 and about pH 8.5, between about pH 4.5 and about pH 8.0, between about pH 4.7 and about pH 7.5, between about pH 4.8 and about pH 9.4, between about pH 4.8 and about pH 9.0, between about pH 4.8 and about pH 8.5, between about pH 4.8 and about pH 8.0, and between about pH 4.8 and about pH 7.5.

In some embodiments of the uses and methods described herein, said designed ankyrin repeat domain comprises an N-terminal capping module, a C-terminal capping module and one or more internal repeat module(s).

In some embodiments of the uses and methods described herein, the designed ankyrin repeat domains may be obtained by substitution of an amino acid, methods to perform such substitutions are well known in the art and include mutagenesis of the cDNA encoding the described repeat domains.

Residues selected for substitutions can be located at randomized or non-randomized positions of the repeat domain. Accordingly, in some embodiments, the substituted residues are selected among residues located at non-randomized positions of said repeat domain. In other embodiments the substituted residues are selected among residues located at randomized positions of said repeat domain. In some embodiments the substituted residues are selected among residues located at randomized and non-randomized positions of said repeat domain.

In other embodiments the substituted residues are selected among all residues comprised in said repeat domain. All residues in this sense shall mean any of the residues located at a randomized or non-randomized position comprised in a designed ankyrin repeat domain described herein. Preferred randomized positions are shown in Table A. Table B shows preferred non-randomized positions of the designed ankyrin repeat domains described herein.

In some embodiments of the uses and methods described herein, the substitutions are only performed in the N-terminal capping module. In some embodiments of the uses and methods described herein, the substitutions are only performed in the C-terminal capping module. In some embodiments of the uses and methods described herein, the substitutions are only performed in the C-terminal and in the in the N-terminal capping modules. In some embodiments of the uses and methods described herein, the substitutions are only performed in the internal repeat module(s).

Examples of conservative and other exemplary amino acid residue substitutions that may occur in designed ankyrin repeat domains and proteins described herein are shown in Table C. In preferred embodiments, the substitute amino acid is not cysteine, glycine, or proline.

In some embodiments of the uses and methods described herein, said designed ankyrin repeat domain comprises:
(a) an N-terminal capping module having the amino acid sequence of SEQ ID NO: 5 or any variant thereof having at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with SEQ ID NO: 5, and/or
(b) a C-terminal capping module having the amino acid sequence of SEQ ID NO: 6 or any variant thereof having at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with SEQ ID NO: 6, and/or
(c) one or more internal repeat module(s) each independently having the amino acid sequence of SEQ ID NO: 7 or any variant thereof having at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with SEQ ID NO: 7.

Accordingly, in one embodiment, said designed ankyrin repeat domain comprises:
(a) an N-terminal capping module having the amino acid sequence of SEQ ID NO: 5 or any variant thereof having at least 90% sequence identity with SEQ ID NO: 5, and/or
(b) a C-terminal capping module having the amino acid sequence of SEQ ID NO: 6 or any variant thereof having at least 90% sequence identity with SEQ ID NO: 6, and/or
(c) one or more internal repeat module(s) each independently having the amino acid sequence of SEQ ID NO: 7 or any variant thereof having at least 90% sequence identity with SEQ ID NO: 7.

In some embodiments of the uses and methods described herein, said designed ankyrin repeat domain comprises:
(a) an N-terminal capping module having the amino acid sequence of SEQ ID NO: 5 or any variant of SEQ ID NO: 5 wherein:
   (i) up to 1, up to 2, up to 3 or up to 4 amino acids selected from the randomized positions corresponding to positions 4, 8, 11 and 12 of SEQ ID NO: 5 are substituted by a different amino acid, and
   (ii) up to 1, up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25 or up to 26 amino acids selected from the non-randomized positions corresponding to positions 1, 2, 3, 5, 6, 7, 9, 10, 13, 14, 15, 16, 17, 18, 19, 20, 21, 22, 23, 24, 25, 26, 27, 28, 29 and 30 of SEQ ID NO: 5 are substituted by a different amino acid, and/or
(b) a C-terminal capping module having the amino acid sequence of SEQ ID NO: 6 or any variant of SEQ ID NO: 6 wherein:
   (i) up to 1, up to 2, up to 3, up to 4 or up to 5 amino acids selected from the randomized positions corresponding to positions 3, 4, 6, 14 and 15 of SEQ ID NO: 6 are substituted by a different amino acid, and
   (ii) up to 1, up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22 or up to 23 amino acids selected from the non-randomized positions corresponding to positions 1, 2, 5, 7, 8, 9, 10, 11, 12, 13, 16, 17, 18, 19, 20, 21, 22, 23, 24, 25, 26, 27 and 28 of SEQ ID NO: 6 are substituted by a different amino acid, and/or
(c) one or more internal repeat module(s) each independently having the amino acid sequence of SEQ ID NO: 7 or any variant of SEQ ID NO: 7 wherein:
   (i) up to 1, up to 2, up to 3, up to 4, up to 5 or up to 6 amino acids selected from the randomized positions corresponding to positions 3, 4, 6, 11 14 and 15 of SEQ ID NO: 7 are substituted by a different amino acid, and
   (ii) up to 1, up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26 or up to 27 amino acids selected from the non-randomized positions corresponding to positions 1, 2, 5, 7, 8, 9, 10, 12, 13, 16, 17, 18, 19, 20, 21, 22, 23, 24, 25, 26, 27, 28, 29, 30, 31, 32 and 33 of SEQ ID NO: 7 are substituted by a different amino acid.

Accordingly, in one embodiment, said designed ankyrin repeat domain comprises:
(a) an N-terminal capping module having the amino acid sequence of SEQ ID NO: 5 or any variant of SEQ ID NO: 5 wherein:
   (i) up to 4 amino acids selected from the randomized positions corresponding to positions 4, 8, 11 and 12 of SEQ ID NO: 5 are substituted by a different amino acid, and
   (ii) up to 15 amino acids selected from the non-randomized positions corresponding to positions 1, 2, 3, 5, 6, 7, 9, 10, 13, 14, 15, 16, 17, 18, 19, 20, 21, 22, 23, 24, 25, 26, 27, 28, 29 and 30 of SEQ ID NO: 5 are substituted by a different amino acid, and/or
(b) a C-terminal capping module having the amino acid sequence of SEQ ID NO: 6 or any variant of SEQ ID NO: 6 wherein:
   (i) up to 5 amino acids selected from the randomized positions corresponding to positions 3, 4, 6, 14 and 15 of SEQ ID NO: 6 are substituted by a different amino acid, and
   (ii) up to 15 amino acids selected from the non-randomized positions corresponding to positions 1, 2, 5, 7, 8, 9, 10, 11, 12, 13, 16, 17, 18, 19, 20, 21, 22, 23, 24, 25, 26, 27 and 28 of SEQ ID NO: 6 are substituted by a different amino acid, and/or
(c) one or more internal repeat module(s) each independently having the amino acid sequence of SEQ ID NO: 7 or any variant of SEQ ID NO: 7 wherein:
   (i) up to 6 amino acids selected from the randomized positions corresponding to positions 3, 4, 6, 11 14 and 15 of SEQ ID NO: 7 are substituted by a different amino acid, and
   (ii) up to 15 amino acids selected from the non-randomized positions corresponding to positions 1, 2, 5, 7, 8, 9, 10, 12, 13, 16, 17, 18, 19, 20, 21, 22, 23, 24, 25, 26, 27, 28, 29, 30, 31, 32 and 33 of SEQ ID NO: 7 are substituted by a different amino acid.

In some embodiments of the uses and methods described herein, said designed ankyrin repeat domain comprises two or more internal repeat modules. In more particular embodiments, said internal repeat modules comprised in said repeat domain have at least 80%, at least 85%, at least 90%, at least 91%, at least 92%, at least 93%, at least 94%, at least 95%, at least 96%, at least 97%, at least 98%, at least 99% or 100% sequence identity between each other. In some embodiments, the residues located at randomized positions of said internal repeat modules comprised in said ankyrin repeat domain are identical between each of said internal repeat modules. In preferred embodiments, said randomized positions correspond to positions 3, 4, 6, 11, 14 and 15 of the internal repeat module, numbered relative to SEQ ID NO: 7.

In some embodiments, said designed ankyrin repeat domain comprises one, two, three, four, five, six, seven, eight or nine internal repeat modules. In preferred embodiments, said designed ankyrin repeat domain comprises two internal repeat modules. In some embodiments, said designed ankyrin repeat domain comprises exactly one, two, three, four, five, six, seven, eight or nine internal repeat modules.

In some embodiments of the uses and methods described herein, the designed ankyrin repeat domain comprises an amino acid sequence selected from the group consisting of (1) SEQ ID NO: 1 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 1. Thus, in a more particular embodiment, said designed repeat domain comprises an amino acid sequence selected from the group consisting of (1) SEQ ID NO: 1 and (2) sequences with at least 80% amino acid sequence identity with SEQ ID NO: 1.

In some embodiments of the uses and methods described herein, the designed ankyrin repeat domain comprises an amino acid sequence selected from the group consisting of (1) SEQ ID NO: 11 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 11. Thus, in a more particular embodiment, said designed repeat domain comprises an amino acid sequence selected from the group consisting of (1) SEQ ID NO: 11 and (2) sequences with at least 80% amino acid sequence identity with SEQ ID NO: 11.

In some embodiments of the uses and methods described herein, the designed ankyrin repeat domain comprises an amino acid sequence selected from the group consisting of (1) SEQ ID NO: 12 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 12. Thus, in a more particular embodiment, said designed repeat domain comprises an amino acid sequence selected from the group consisting of (1) SEQ ID NO: 12 and (2) sequences with at least 80% amino acid sequence identity with SEQ ID NO: 12.

In some embodiments of the uses and methods described herein, the designed ankyrin repeat domain comprises an amino acid sequence selected from the group consisting of (1) SEQ ID NO: 13 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 13. Thus, in a more particular embodiment, said designed repeat domain comprises an amino acid sequence selected from the group consisting of (1) SEQ ID NO: 13 and (2) sequences with at least 80% amino acid sequence identity with SEQ ID NO: 13.

In some embodiments of the uses and methods described herein, the designed ankyrin repeat domain comprises an amino acid sequence selected from the group consisting of (1) SEQ ID NO: 14 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 14. Thus, in a more particular embodiment, said designed repeat domain comprises an amino acid sequence selected from the group consisting of (1) SEQ ID NO: 14 and (2) sequences with at least 80% amino acid sequence identity with SEQ ID NO: 14.

In some embodiments of the uses and methods described herein, the designed ankyrin repeat domain comprises an amino acid sequence selected from the group consisting of (1) SEQ ID NO: 15 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 15. Thus, in a more particular embodiment, said designed repeat domain comprises an amino acid sequence selected from the group consisting of (1) SEQ ID NO: 15 and (2) sequences with at least 80% amino acid sequence identity with SEQ ID NO: 15.

In some embodiments of the uses and methods described herein, the designed ankyrin repeat domain comprises an amino acid sequence selected from the group consisting of (1) SEQ ID NO: 16 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 16. Thus, in a more particular embodiment, said designed repeat domain comprises an amino acid sequence selected from the group consisting of (1) SEQ ID NO: 16 and (2) sequences with at least 80% amino acid sequence identity with SEQ ID NO: 16.

In some embodiments of the uses and methods described herein, the designed ankyrin repeat domain comprises an amino acid sequence selected from the group consisting of (1) SEQ ID NO: 17 and (2) sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% amino acid sequence identity with SEQ ID NO: 17. Thus, in a more particular embodiment, said designed repeat domain comprises an amino acid sequence selected from the group consisting of (1) SEQ ID NO: 17 and (2) sequences with at least 80% amino acid sequence identity with SEQ ID NO: 17.

In some embodiments of the uses and methods described herein, the designed ankyrin repeat domain or the recombinant protein comprising a designed ankyrin repeat domain is administered to the subject in form of a pharmaceutical composition comprising said repeat domain or said recombinant protein and optionally at least one pharmaceutically acceptable carrier or diluent. In some embodiments of the uses and methods described herein, said pharmaceutical composition also comprises the therapeutic and/or diagnostic agent.

Such pharmaceutical compositions may be prepared using methods known in the art, as also described herein.

In another aspect, the invention provides a method of reducing accumulation of a therapeutic and/or diagnostic agent in the kidney of a subject treated with said agent, the method comprising the step of administering to said subject an effective amount of the designed ankyrin repeat domain, the recombinant protein or the pharmaceutical composition of the invention.

In some embodiments, the invention provides a method for reducing accumulation of a therapeutic and/or diagnostic agent in the kidney of a subject treated with said agent, the method comprising administering to said subject the designed ankyrin repeat domain of the invention in an amount effective to reduce accumulation of said therapeutic and/or diagnostic agent in the kidney.

Further provided is the designed ankyrin repeat domain, the recombinant protein, or the pharmaceutical composition of the invention for use in a method of reducing accumulation of a therapeutic and/or diagnostic agent in the kidney of a subject treated with said agent, the method comprising the step of administering to said subject an effective amount of the designed ankyrin repeat domain, the recombinant protein or the pharmaceutical composition of the invention to reduce accumulation of said agent in the kidney.

In one embodiment, the invention relates to the use of the designed repeat domain, the recombinant protein or the pharmaceutical composition according to the present invention for reducing accumulation of a therapeutic and/or diagnostic agent in the kidney of a subject being treated with said agent, wherein the designed ankyrin repeat domain, the recombinant protein or the pharmaceutical composition according to the invention is administered to said subject in an effective amount to reduce accumulation of said therapeutic and/or diagnostic agent in the kidney

In one embodiment, the invention relates to the use of the designed ankyrin repeat domain, recombinant protein or pharmaceutical composition of the invention, for manufacturing of a medicament.

In one embodiment, the invention relates to the use of the designed ankyrin repeat domain, recombinant protein or pharmaceutical composition of the invention, for manufacturing of a medicament for reducing accumulation of a therapeutic and/or diagnostic agent in the kidney of a subject being treated with said agent.

In some embodiments of the uses and methods described herein said recombinant protein has a molecular weight of 69 kDa or less, 65 kDa or less, 60 kDa or less, 55 kDa or less, 50 kDa or less, 45 kDa or less, 40 kDa or less, 35 kDa or less, or 30 kDa or less.

In the context of the invention, the terms “medical condition”, “disease” and “disorder” are used interchangeably and include but are not limited to cancer. In one preferred embodiment, said medical condition is a cancer.

In some further embodiments, the methods and uses of the invention may be used in radiopharmaceutical therapy or diagnostics. Exemplary approaches and indications are for instances disclosed in Sgouros, George, et al. “Radiopharmaceutical therapy in cancer: clinical advances and challenges.” Nature Reviews Drug Discovery 19.9 (2020): 589-608.

In some alternative embodiments, methods and uses of the invention may be used in therapeutic and/or diagnostic approaches for which also antibody-drug conjugates may be used. Such approaches and indications are for instance disclosed in Drago, Joshua Z., Shanu Modi, and Sarat Chandarlapaty. Nature Reviews Clinical Oncology 18.6 (2021) and Tarantino, Paolo, et al., CA: a cancer journal for clinicians 72.2 (2022): 165-182.

The invention is not restricted to the particular embodiments described in the Examples.

### EXAMPLES

### Materials

Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). Inducible E. coli expression strains were used for cloning and protein production, e.g. E. coli XL1-blue (Stratagene, USA) or BL21 (Novagen, USA). TEV protease was from Sigma-Aldrich (USA). Double-stranded gene fragments (eBlocks) were obtained from IDT (US). Maleimide DTPA was purchased from Chematech, metal-free PBS was purchased from VWR and Chelex 100 chelating resins were purchased from BioRad.

### Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989, New York).

### Designed Ankyrin Repeat Protein Libraries

Methods to generate designed ankyrin repeat protein libraries have been described, e.g. in U.S. Pat. No. 7,417,130; Binz et al. 2003, loc. cit.; Binz et al. 2004, loc. cit. By such methods designed ankyrin repeat protein libraries having randomized ankyrin repeat modules and/or randomized capping modules can be constructed. For example, such libraries could accordingly be assembled based on a fixed N-terminal capping module or a randomized N-terminal capping module, one or more randomized repeat modules, and a fixed C-terminal capping module or a randomized C-terminal capping module (see, e.g., the N-terminal capping modules and C-terminal capping modules provided in WO2021116462 and WO2021116469. Preferably, such libraries are assembled to not have any of the amino acids C, G, M, N (in front of a G residue) and P at randomized positions of repeat or capping modules.

Furthermore, such randomized modules in such libraries may comprise additional polypeptide loop insertions with randomized amino acid positions. Examples of such polypeptide loop insertions are complement determining region (CDR) loop libraries of antibodies or de novo generated peptide libraries. For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23 (30), 3929-3938, 2004) as guidance. In analogy to this ankyrin repeat domain where ten amino acids are inserted in the beta-turn present close to the border of two ankyrin repeats, ankyrin repeat protein libraries may contain randomized loops (with fixed and randomized positions) of variable length (e.g. 1 to 20 amino acids) inserted in one or more beta-turns of an ankyrin repeat domain.

An N-terminal capping module of an ankyrin repeat protein library preferably possesses the RILLAA, RILLKA or RELLKA motif and any such C-terminal capping module of an ankyrin repeat protein library preferably possesses the KLN, KLA or KAA motif.

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V2O, 3UXG, 3TWQ-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

The nomenclature used to define the positions inside the repeat units and modules is based on Binz et al. 2004, loc. cit. with the modification that borders of the ankyrin repeat modules and ankyrin repeat units are shifted by one amino acid position. For example, position 1 of an ankyrin repeat module of Binz et al. 2004 (loc. cit.) corresponds to position 2 of an ankyrin repeat module of the current disclosure and consequently position 33 of an ankyrin repeat module of Binz et al. 2004, loc. cit. corresponds to position 1 of a following ankyrin repeat module of the current disclosure.

The experimental conditions for some examples are also further described in WO2012069654, WO2016156596 and WO2021116462.

### Example 1: Kidney Accumulation of Radiolabeled DARPins, “Cold” Competitor DARPin Saturation Study

This example describes experiments that were performed to investigate the kidney accumulation of radiolabeled DARPin when co-injected with an excess of “cold” competitor DARPin, wherein the “cold” competitor has the same DARPin sequence as the “hot” radiolabeled DARPin. The “cold” competitor is not radiolabeled, not coupled to the chelator (DTPA) and has a N-terminal His tag of SEQ ID NO: 9.

DARPins having a defined amino acid sequence can be produced by gene synthesis of a corresponding reverse translated nucleic acid sequence, subcloning into an appropriate expression vector of an expression system (e.g. an E. coli expression system), expression and purification of the protein. Such methods are known to the person skilled in the art.

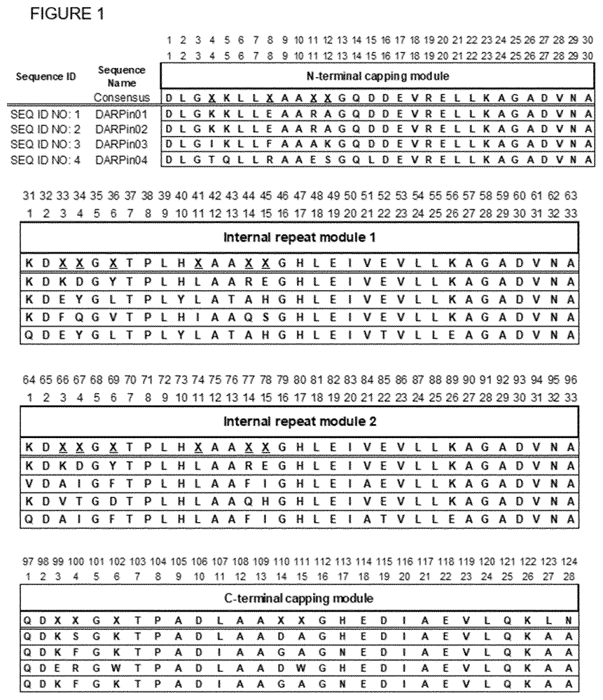

The three DARPins sequences used for this study are shown in Table 1 (and FIG. 1). DARPin02 and DARPin03 were described previously in WO2018054971 and DARPin01 in WO2020245746 & WO2020245175. These DARPins and their radiolabeled counterparts were produced and characterized as described in the following paragraphs.

TABLE 1

| Name | SEQ ID | pl |
|---|---|---|
| DARPin01 | NO: 1 | 4.93 |
| DARPin02 | NO: 2 | 4.65 |
| DARPin03 | NO: 3 | 4.64 |

### 1.1 Construction, Expression and Purification of DARPins

To produce radiolabeled DARPins based on SEQ ID NOs: 1, 2 and 3, the DNA encoding each of said designed ankyrin repeat domains was first cloned into a pQE (QIAgen, Germany) based expression vector providing an N-terminal 6×His-tag to facilitate simple protein purification. Proteins comprising one of SEQ ID NOS: 1, 2 and 3, respectively, and additionally having a His-TEV tag (SEQ ID NO: 8) fused to their N-termini and a GSGSC tag (SEQ ID NO: 10) fused to their C-termini were expressed in E. coli, and purified over an IMAC column before further purification over a HiLoad 26/600 Superdex 200 column. Main fractions were pooled and 4 mL of DARPin (different concentrations) were digested with 290 μL of TEV (Sigma Aldrich, >3kU/mg TEV) at RT (for 2 h), then at 4° C. (overnight). Samples were taken after 2 h and after overnight digestion and analyzed on an SDS-PAGE to assess the degree of cleavage. Non-cleaved DARPins still containing the His-tag as well as the His-tagged TEV protease were removed by incubating for 2 h with 5 mL of IMAC resin on a roller shaker, before centrifugation and removal of the IMAC resins by decanting and filtration. Supernatant/flow-through was purified over a size-exclusion chromatography step, before up-concentration. Final purified samples were stored in PBS.

To produce “cold” competitor DARPins based on SEQ ID NOs: 1, 2 and 3, these designed ankyrin repeat domains were expressed in E. coli cells and purified using a His-tag according to standard protocols. 65 ml of stationary overnight cultures (TB, 1% glucose, 50 mg/l of ampicillin; 37° C.) were used to inoculate 2000 ml cultures (TB, 50 mg/l ampicillin, 37° C.). At an absorbance of 1.0 to 1.5 at 600 nm, the cultures were induced with 1 mM IPTG and incubated at 37° C. for 5 h while shaking. The cultures were centrifuged, and the resulting pellets were re-suspended in 25 ml of TBS500 (50 mM Tris-HCl, 500 mM NaCl, pH 8) and stored at −20° C., before they were thawed, mixed with 50 KU DNase/ml and 1 mg/ml of lysozyme and lysed by sonication). Following the lysis, the samples comprising SEQ ID NO: 1 and SEQ ID NO: 3 were heat treated (62.5° C. for 30 min), whereas sample comprising SEQ ID NO:2 was not heat treated and directly taken to the next processing step. All three samples were then centrifuged and the supernatant was collected and filtrated. Proteins were purified over an immobilized metal affinity column (IMAC) followed by size exclusion chromatography (HiLoad 26/600 Superdex 200 column) using an Aekta Express system. Highly soluble ankyrin repeat proteins were purified from E. coli culture (up to 200 mg ankyrin repeat protein per liter of culture) with a purity >95% as estimated from 4-12% SDS-PAGE. Detailed methods for the production and purification of proteins are well known to the practitioner in the art.

### 1.2 Size-Exclusion Chromatography (SEC) Analysis

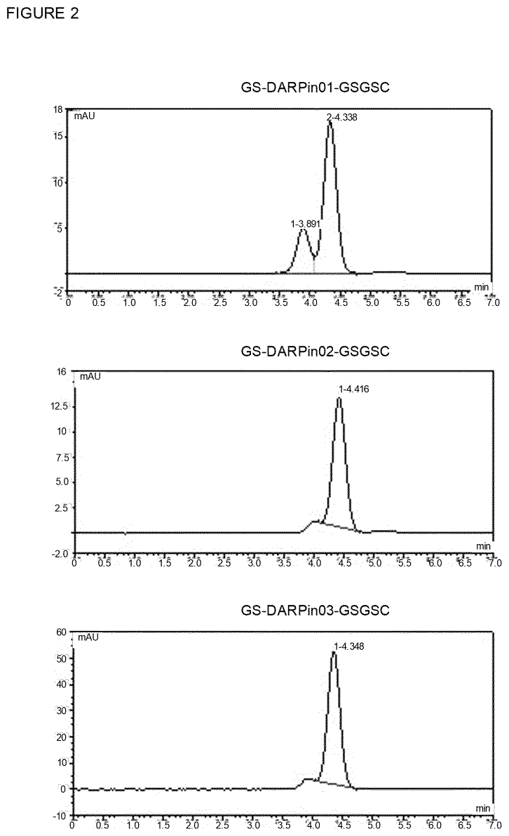

Samples for subsequent production of radiolabeled DARPins were analyzed on a GE Superdex Increase 200 150/5 column on an Agilent 1200 HPLC system in PBS at 0.5 ml/min flow rate. Of each protein, 0.1 ml at 100 micromolar concentration were analyzed. Proteins comprising one of SEQ ID NOs: 1, 2 and 3, respectively, and a C-terminal GSGSC tag (SEQ ID NO: 10) elute as partially dimeric peaks due to oxidation of the C-terminal cysteine to form homo-dimers. At least 95% of the area under the curve corresponded to the combined monomer+dimer fraction. Results are shown in FIG. 2. These results indicate that the proteins are biophysically well-behaved.

SEC analysis of the “cold” competitor DARPins based on SEQ ID NO: 1, 2 and 3 (not shown) was performed according to standard protocol known to the skilled person in the art.

### 1.3 DTPA Coupling to C-Terminal Cysteine of DARPin and ¹¹¹In Loading

His-tag free DARPins of SEQ ID NOs: 1, 2 and 3 containing the C-terminal Cys were first reduced by incubating a protein solution of approximatively 5 mg/mL with a 10-fold excess of 0.5 M TCEP (pH adjusted to 7.6). The reaction was shaken for 4 h at room temperature. Subsequently, reduced DARPin solution was mixed with 0.5 M EDTA at a 1:1 molar ratio and stirred for 15 min. Then, a 5-fold molar excess of 50 mM maleimide-DTPA (2,2′-(1-carboxy-2-(carboxymethyl)-13-(2,5-dioxo-2,5-dihydro-1H-pyrrol-1-yl)-10-oxo-2,5,8,11-tetraazatridecane-5,8-di-yl)diacetic acid) dissolved in DMSO was added and stirred for 1 h at room temperature. Probes of the sample were taken and used for analysis via ESI-MS to assess the coupling efficiency of the reaction. If coupling efficiency was >90%, samples were desalted over PD-10 column according to the manufacturer's instruction into metal-free PBS. Then, the protein solution was concentrated and diluted with metal-free PBS for three times over Amicon Ultra-15 centrifugal filters (3K) to desalt the protein further. The final concentration was determined by UV-absorption and a probe for another ESI-MS analysis was taken and measured.

To load the DTPA-coupled DARPin with the radioisotope indium-111, 10-20 MBq of ¹¹¹In was mixed with 5 μL of 1 M ammonium acetate buffer, then the DTPA-coupled DARPin was added (50-200 μL of approximatively 1 mg/mL DARPin-DTPA construct). The solution was stirred at 37.5° C. for 18 hours, then 1 μL of 0.5 M EDTA was added to complex non-bound ¹¹¹In. The labeling efficiency of ¹¹¹In-labeled DARPin was checked by HPLC, only if the labeling yield was <90%, the labeled protein was purified over a PD-10 desalting column to remove free ¹¹¹In. Finally, to reach a specific activity of 7500 Bq/μg of DARPin, non-radioactive loaded DTPA-DARPin was added to adjust for the final concentration. The entire process of generating the radioactively loaded DTPA-coupled DARPins is illustrated in FIG. 3. Table 2 summarizes features of the produced radiolabeled DARPins.

TABLE 2

| radiolabeled | | |
|---|---|---|
| DARPin | | |
| No. | Description | Sequence |
| 01 | GS-DARPin01- | SEQ ID NO: 1 with a C-terminal GSGSC |
| | GSGSC- | tag of SEQ ID NO: 10 and “GS” residues |
| | DTPA-¹¹¹In | at the N-terminal |
| 02 | GS-DARPin02- | SEQ ID NO: 2 with a C-terminal GSGSC |
| | GSGSC- | tag of SEQ ID NO: 10 and “GS” residues |
| | DTPA-¹¹¹In | at the N-terminal |
| 03 | GS-DARPin03- | SEQ ID NO: 3 with a C-terminal GSGSC |
| | GSGSC- | tag of SEQ ID NO: 10 and “GS“ residues |
| | DTPA-¹¹¹In | at the N-terminal |

### 1.4 SPR Single Trace Analysis

DARPin02 and DARPin03 have a target binding specificity for HER2. This binding specificity is maintained upon DTPA coupling as assessed by surface plasmon resonance (SPR) analysis.

All SPR data were generated using a Bruker Sierra SPR-32 instrument with PBS-T (0.005% Tween 20) as running buffer. A new Bruker BTC Chip was conditioned according to the manufacturer's protocol. The chip was coated with biotinylated target (bio-HER2) to reach a signal intensity of approximatively 600 RU. All analytes (500 nM) were injected in succession for 120 s, dissociation was recorded for 180 s (25 ul/min). Each injection was followed by a regeneration step with glycine pH 2.0 for 60 s. The data was double referenced (control spot and buffer injection) and fitted to a 1:1 Langmuir model.

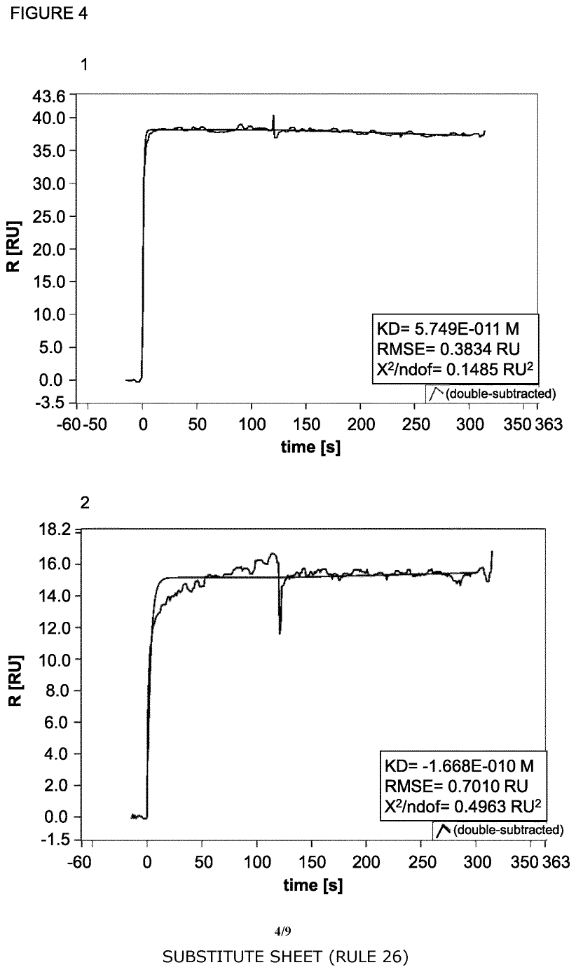

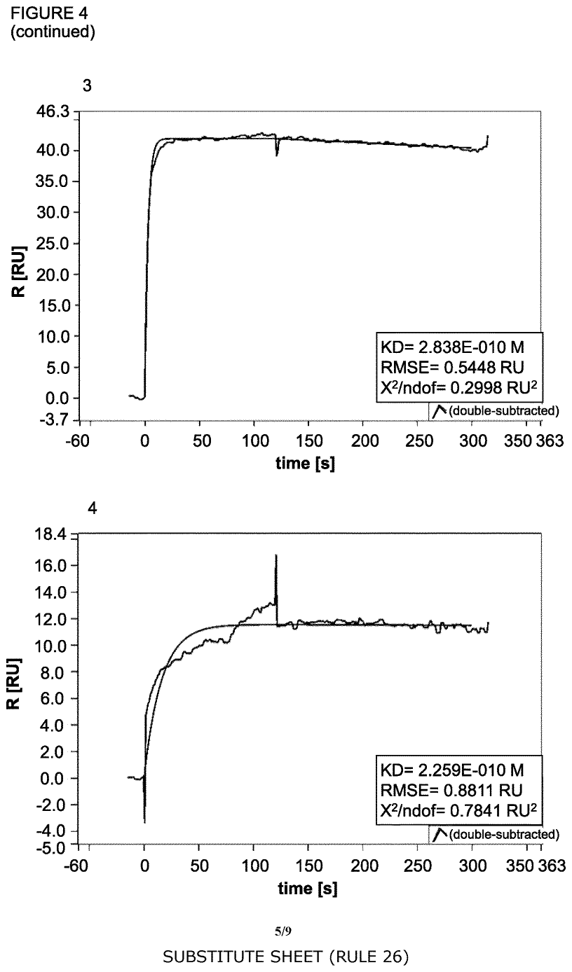

SPR curves are shown in FIG. 4 where plots 1 and 2 show the profiles of the non DTPA-coupled HER2-binders DARPin02 and DARPin03 (SEQ ID NOS: 2 and 3), respectively. Plots 3 and 4 show the profiles of the DTPA coupled counterparts GS-DARPin02-GSGSC-DTPA and GS-DARPin03-GSGSC-DTPA respectively. The GSGSC tag (SEQ ID NO: 10) was fused to the C-terminal end of the DARPins.

### 1.5 Kidney Accumulation Study in Wild-Type Mice

Radiolabeled DARPins 01, 02 and 03 were injected (approximately 150 KBq, 1 mg/kg BW) with or without a 50-molar-fold excess of “cold” DARPins (Cold-DARPin01, Cold-DARPin02 and Cold-DARPin03 respectively) into the tail vein of wild-type Balb/c mice (females, 7 weeks of age, CRL), as detailed in Table 3. The compounds were formulated in PBS+0.05% Tween 20.

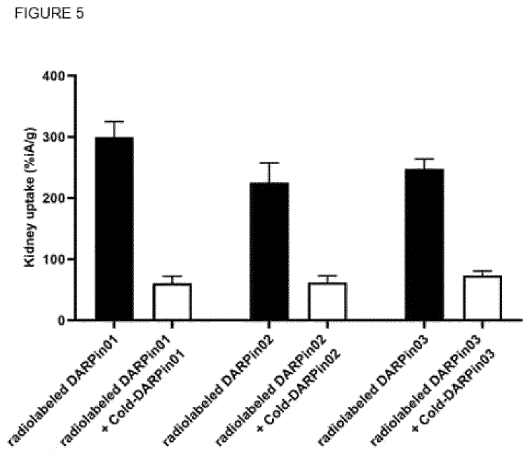

Kidney accumulation was measured at 4 h post-injection. Mice were euthanized by CO₂ inhalation and cervical dislocation. Kidneys were extracted, weighed and the radioactivity was determined with a γ counter (Packard Cobra II Gamma D5010, GMI, USA). The data are expressed as injected activity per gram of tissue mass (% IA/g) and shown in FIG. 5.

For autoradiography imaging, kidneys were embedded in OCT and frozen at −80 C. 24 h after collection frozen kidneys sections were prepared on a cryostat and mounted on glass slides. The sections were placed in a X-ray cassette and exposed to phosphor screens for 45 min.

TABLE 3

| | | | Mice | Dosing |
|---|---|---|---|---|
| Group | Treatment | Construct details | number | mg/kg |
| 1 | radiolabeled | GS-DARPin01-GSGSC- | 4 | 1 |
| | DARPin01 | DTPA-¹¹¹In | | |
| 2 | radiolabeled | GS-DARPin01-GSGSC- | 4 | 1 |
| | DARPin01 | DTPA-¹¹¹In | | |
| | Cold-DARPin01 | MRGSHis6GS-DARPin01 | | 50 |
| 3 | radiolabeled | GS-DARPin02-GSGSC- | 4 | 1 |
| | DARPin02 | DTPA-¹¹¹In | | |
| 4 | radiolabeled | GS-DARPin02-GSGSC- | 4 | 1 |
| | DARPin02 | DTPA-¹¹¹In | | |
| | Cold-DARPin02 | MRGSHis6GS-DARPin02 | | 50 |
| 5 | radiolabeled | GS-DARPin03-GSGSC- | 4 | 1 |
| | DARPin03 | DTPA-¹¹¹In | | |
| 6 | radiolabeled | GS-DARPin03-GSGSC- | 4 | 1 |
| | DARPin03 | DTPA-¹¹¹In | | |
| | Cold-DARPin03 | MRGSHis6GS-DARPin03 | | 50 |

As shown in FIG. 5, for all three tested radiolabeled DARPins, kidney uptake in the blocked groups (2, 4 and 6) is highly reduced compared to the non-blocked groups (1, 3 and 5), respectively.

Table 4 shows the reduction in kidney uptake (in percent) in this experiment, when comparing the administrations with radiolabeled DARPin alone to the administrations with radiolabeled DARPin supplemented with a 50-fold excess of “cold” DARPin. These percentage reduction values are computed according to the formula: percent reduction=100−(X/Y*100), where X is the accumulation measured in the blocked group, and Y is the accumulation measured in the non-blocked group.

TABLE 4

| | | Reduction in kidney |
|---|---|---|
| Blocked (50-fold cold | | uptake blocked |
| DARPin) | non-blocked | vs. non-blocked |
| radiolabeled DARPin01 + | radiolabeled DARPin01 | 80% |
| Cold-DARPin01 | | |
| radiolabeled DARPin02 + | radiolabeled DARPin02 | 72% |
| Cold-DARPin02 | | |
| radiolabeled DARPin02 + | radiolabeled DARPin03 | 70% |
| Cold-DARPin03 | | |

### Example 2: Kidney Accumulation of Radiolabeled DARPins, “Cold” Mock DARPin Saturation Study

This example describes experiments that were performed to investigate the kidney accumulation of radiolabeled DARPin when co-injected with an excess of “cold” DARPin, wherein the “cold” DARPin differs from the “hot” DARPin in structure and binding specificity. The “cold” competitor is not radiolabeled, not coupled to the chelator (DTPA) and has a N-terminal His tag of SEQ ID NO: 9.

The DARPins used in this experiment are based on three DARPin sequences: DAPRin04, DARPin02 and DARPin01. DARPin04 has been structurally engineered and was shown (EP22188160.0) to induce a lower renal accumulation when administered as radiolabeled DARPin compared to its parental non-engineered DARPin. This parental DARPin corresponds to DARPin02 used in Example 1. More specifically, DARPin04 has a lower isoelectric point (pI) and has a lower percentage of basic amino acids when compared to DARPin02; the details of DARPin04 are shown in Table 5 (and FIG. 1). Both DARPin02 and DARPin04 specifically bind to HER2. The cold DARPin used in this experiment is Cold-DARPin01, which is the same molecule as in Example 1 and is structurally distinct from DARPin04.

TABLE 5

| Name | SEQ ID | pl |
|---|---|---|
| DARPin04 | NO: 4 | 4.26 |

DAPRin04, DARPin02 and Cold-DARPin01 have been produced as described in sections 1.1 to 1.3 of Example 1. The radiolabeled DARPin04 resulting from the ¹¹¹In labeling is shown in Table 6.

TABLE 6

| radiolabeled | | |
|---|---|---|
| DARPin No. | Description | Sequence |
| 04 | GS-DARPin04- | SEQ ID NO: 4 with a |
| | GSGSC-DTPA-¹¹¹In | C-terminal GSGSC tag |
| | | of SEQ ID NO: 10 |
| | | and “GS” residues at |
| | | the N-terminal |

### 2.1 Kidney Accumulation Study in Tumor Bearing CD1-Nude Mice

Radiolabeled DARPin04 was injected (approximately 150 KBq, 1 mg/kg BW) with or without a 50-molar-fold excess of “cold” DARPin (Cold-DARPin01) into the tail vein of HER2-expressing SKOV3ip tumor bearing mice (females, 9-12 weeks of age, Crl:CD1-Foxn1nu), as detailed in Table 7. Parental DARPin02 was similarly injected, without a co-injection of cold DARPin. The compounds were formulated in PBS+0.05% Tween 20.

Tumor cells (5×10⁶, in PBS) were implanted subcutaneously into the flank of the mice. Two mice groups were considered, based on the size of the tumor at the time of injection. A first group in which mice were randomized into the different treatment groups and i.v. injected with ¹¹¹In-labelled DARPins two weeks after implantation at a tumor volume of approx. 180 mm³; a second group in which mice were randomized into the different treatment groups and i.v. injected with ¹¹¹In-labelled DARPins three weeks after implantation at a tumor volume of approx. 360 mm³. Tumor and organs were collected and the % ID/g (or % IA/g) was determined.

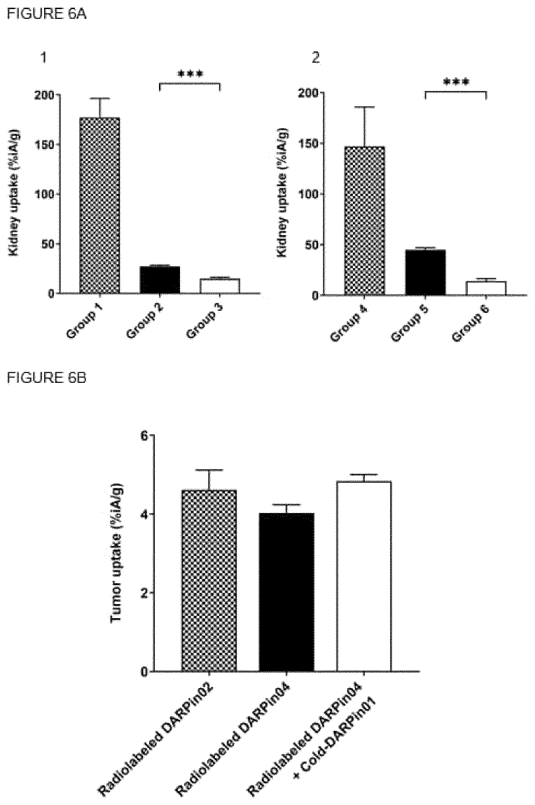

Kidney and tumor accumulation was measured at 4 h post-injection. Mice were euthanized by CO₂ inhalation and cervical dislocation. Kidneys and tumors were extracted, weighed and the radioactivity was determined with a γ counter (Packard Cobra II Gamma D5010, GMI, USA). The data are expressed as injected activity per gram of tissue mass (% IA/g) and shown in FIG. 6.

TABLE 7

| | | | Mice | Mice | |
|---|---|---|---|---|---|
| | | | number, | number, | |
| | | | 180 mm³ | 360 mm³ | Dosing |
| Group | Treatment | Construct details | tumor | tumor | mg/kg |
| 1 | radiolabeled | GS-DARPin02-GSGSC-DTPA-¹¹¹In | 6 | — | 1 |
| | DARPin02 | | | | |
| 2 | radiolabeled | GS-DARPin04-GSGSC-DTPA-¹¹¹In | 6 | — | 1 |
| | DARPin04 | | | | |
| 3 | radiolabeled | GS-DARPin04-GSGSC-DTPA-¹¹¹In | 6 | — | 1 |
| | DARPin04 | | | | |
| | Cold-DARPin01 | MRGSHis6GS-DARPin01 | | | 50 |
| 4 | radiolabeled | GS-DARPin02-GSGSC-DTPA-¹¹¹In | — | 6 | 1 |
| | DARPin02 | | | | |
| 5 | radiolabeled | GS-DARPin04-GSGSC-DTPA-¹¹¹In | — | 6 | 1 |
| | DARPin04 | | | | |
| 6 | radiolabeled | GS-DARPin04-GSGSC-DTPA-¹¹¹In | — | 6 | 1 |
| | DARPin04 | | | | |
| | Cold-DARPin01 | MRGSHis6GS-DARPin01 | | | 50 |

As can be seen in FIG. 6A, a reduction in kidney accumulation was observed in both “cold” mock DARPin-blocked groups (Group 3 and 6) compared to the non-blocked groups (Group 2 and 5) respectively. These data further show that a cumulative effect of the DARPin engineering and the cold blocking approaches is observed on the kidney accumulation of the radiolabeled DARPins. More specifically, a reduction of 84.74% (Group2 vs Group1) and 70% (Group5 vs Group4) can be attributed to the engineering of DARPin04 compared to DARPin02. As shown in Table 8, combining the co-administration of cold blocker (Cold-DARPin01) with the engineering of DARPin04 further decreases the kidney accumulation of radiolabeled DARPin04 by 44.44% (Group3 vs Group2) and 66% (Group6 vs Group5).

Table 8 shows the reduction in kidney uptake (in percent) in this experiment, when comparing the administrations with radiolabeled DARPin04 alone to the administrations with radiolabeled DARPin04 supplemented with a 50-fold excess of cold mock DARPin (Cold-DARPin01). These percentage reduction values are computed as in section 1.5 of Example 1.

TABLE 8

| | | Reduction in kidney |
|---|---|---|
| Blocked (50-fold cold | | uptake blocked |
| DARPin) | non-blocked | vs. non-blocked |
| radiolabeled DARPin04 + | radiolabeled DARPin04 | 44.44% |
| Cold-DARPin01 (Group 3) | (Group 2) | |
| radiolabeled DARPin04 + | radiolabeled DARPin04 | 66% |
| Cold-DARPin01 (Group 6) | (Group 5) | |

The impact of the cold DARPin co-injection on tumor accumulation of the HER2-specific DARPins has been evaluated. Accumulation of radioactivity in the big and small volume tumors was quantified similarly as for the kidney. The difference in accumulation is negligible, as shown in FIG. 6B (big tumors and small tumors are pooled). Based on this data, the co-injection of cold-DAPRin01 does not impair the tumor accumulation of the radiolabeled DARPins, while providing significant reduction of radiolabeled DARPin accumulation in the kidney.

Table 9 further shows the tumor to kidney ratio measured in this experiment. The cold DARPin co-injection provides a 3-fold increase of the tumor to kidney ratio compared to the DARPin injection without cold DARPin co-injection.

TABLE 9

| Samples | Tumor to kidney ratio |
|---|---|
| radiolabeled DARPin02 | 1:35 |
| radiolabeled DARPin04 | 1:9 |
| radiolabeled DARPin04 + Cold-DARPin01 | 1:3 |

### Example 3: Kidney Accumulation of Radiolabeled DARPins, “Cold” DARPin Saturation Study with Additional Blocker Variants

A kidney accumulation study in tumor bearing mice (HER2-expressing SKOV3ip tumor) was performed following the experimental setup and conditions described in Example 2 to test additional cold blocker DARPin variants. The hot 111In radiolabeled DARPin used in this experiment is DARPin02, also described in the previous examples.

Next to DARPin01, seven variants of DARPin01, i.e. DARPin05, DARPin06, DARPin07, DARPin08, DARPin09, DARPin10 and DARPin11 were designed and tested as cold blocker when co-injected with radiolabeled DARPin02. The details of the variants DARPin05 to DARPin11 are shown in Table 10.

TABLE 10

| Name | SEQ ID |
|---|---|
| DARPin05 | NO: 11 |
| DARPin06 | NO: 12 |
| DARPin07 | NO: 13 |
| DARPin08 | NO: 14 |
| DARPin09 | NO: 15 |
| DARPin10 | NO: 16 |
| DARPin11 | NO: 17 |

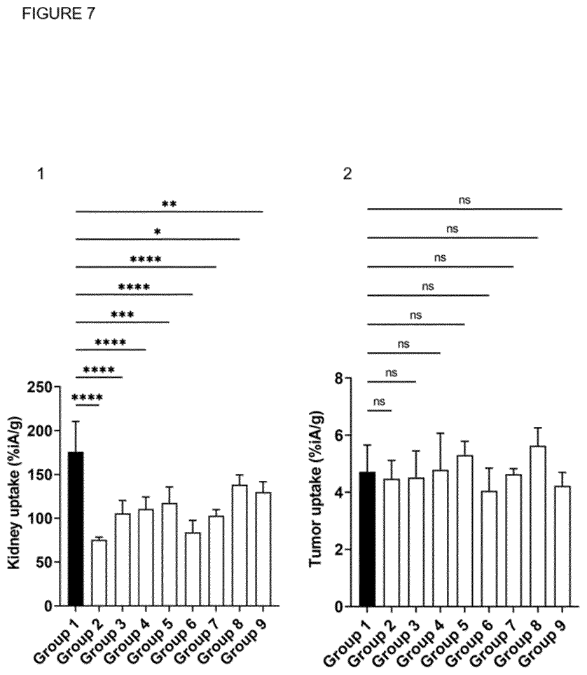

Injections were performed i.v. once tumor volume reached 350 mm³. Kidney and tumor accumulations were measured at 4 h post-injection. The data are expressed as mean injected activity per gram of tissue mass (% IA/g) and shown in FIG. 7. The details of the treatment groups and dosing are shown in table 11.

TABLE 11

| | | | Mice | Dosing |
|---|---|---|---|---|
| Group | Treatment | Construct details | number | mg/kg |
| 1 | radiolabeled | GS-DARPin02-GSGSC- | 4 | 1 |
| | DARPin02 | DTPA-¹¹¹In | | |
| 2 | radiolabeled | GS-DARPin02-GSGSC- | 4 | 1 |
| | DARPin02 | DTPA-¹¹¹In | | |
| | Cold-DARPin01 | MRGSHis6GS-DARPin01 | | 50 |
| 3 | radiolabeled | GS-DARPin02-GSGSC- | 4 | 1 |
| | DARPin02 | DTPA-¹¹¹In | | |
| | Cold-DARPin05 | MRGSHis6GS-DARPin05 | | 50 |
| 4 | radiolabeled | GS-DARPin02-GSGSC- | 4 | 1 |
| | DARPin02 | DTPA-¹¹¹In | | |
| | Cold-DARPin06 | MRGSHis6GS-DARPin06 | | 50 |
| 5 | radiolabeled | GS-DARPin02-GSGSC- | 4 | 1 |
| | DARPin02 | DTPA-¹¹¹In | | |
| | Cold-DARPin07 | MRGSHis6GS-DARPin07 | | 50 |
| 6 | radiolabeled | GS-DARPin02-GSGSC- | 4 | 1 |
| | DARPin02 | DTPA-¹¹¹In | | |
| | Cold-DARPin08 | MRGSHis6GS-DARPin08 | | 50 |
| 7 | radiolabeled | GS-DARPin02-GSGSC- | 4 | 1 |
| | DARPin02 | DTPA-¹¹¹In | | |
| | Cold-DARPin09 | MRGSHis6GS-DARPin09 | | 50 |
| 8 | radiolabeled | GS-DARPin02-GSGSC- | 4 | 1 |
| | DARPin02 | DTPA-¹¹¹In | | |
| | Cold-DARPin10 | MRGSHis6GS-DARPin10 | | 50 |
| 9 | radiolabeled | GS-DARPin02-GSGSC- | 4 | 1 |
| | DARPin02 | DTPA-¹¹¹In | | |
| | Cold-DARPin11 | MRGSHis6GS-DARPin11 | | 50 |

As shown in FIG. 7 plot 1, a significant reduction in kidney accumulation was observed in all cold DARPin-blocked groups (Group 2 to 9) compared to the non-blocked control group (Group 1) respectively (ANOVA, F (8, 27)=13.42, p<0.0001 and Dunnett's test). The biggest kidney accumulation reduction is observed for Group 2 in which Cold-DARPin01 was used. The other tested variants also lead to a reduction in kidney accumulation, nevertheless not reaching the level of Cold-DARPin01.

Table 12 shows the reduction in kidney uptake (in percent) in this experiment, when comparing the administrations with radiolabeled DARPin02 alone to the administrations with radiolabeled DARPin02 supplemented with a 50-fold excess of respective Cold-DARPin01 and Cold-DARPin05 to Cold-DARPin11. These percentage reduction values are computed as in section 1.5 of Example 1.

TABLE 12

| | | Reduction in |
|---|---|---|
| Blocked (50-fold cold | | kidney uptake blocked |
| DARPin) | non-blocked | vs. non-blocked |
| radiolabeled DARPin02 + | radiolabeled DARPin02 | 57.09% |
| Cold-DARPin01 | | |
| radiolabeled DARPin02 + | radiolabeled DARPin02 | 39.99% |
| Cold-DARPin05 | | |
| radiolabeled DARPin02 + | radiolabeled DARPin02 | 37.15% |
| Cold-DARPin06 | | |
| radiolabeled DARPin02 + | radiolabeled DARPin02 | 33.30% |
| Cold-DARPin07 | | |
| radiolabeled DARPin02 + | radiolabeled DARPin02 | 52.32% |
| Cold-DARPin08 | | |
| radiolabeled DARPin02 + | radiolabeled DARPin02 | 41.51% |
| Cold-DARPin09 | | |
| radiolabeled DARPin02 + | radiolabeled DARPin02 | 21.35% |
| Cold-DARPin10 | | |
| radiolabeled DARPin02 + | radiolabeled DARPin02 | 26.20% |
| Cold-DARPin11 | | |

As in Example 2, the impact of the cold DARPin co-injection on tumor accumulation of the HER2-specific DARPin was evaluated. Accumulation of radioactivity in the tumors was quantified similarly as for the kidney. The difference in accumulation between groups is negligible (ANOVA, F (8, 27)=1.674, p=0.151 and Dunnett's test), as shown in FIG. 7 plot 2. Based on this data, the co-injection of cold-DAPRin01 or cold-DAPRin05 to cold-DAPRin11 does not impair the tumor accumulation of the radiolabeled DARPins, while providing significant reduction of radiolabeled DARPin accumulation in the kidney. Table 13 shows the corresponding average tumor to kidney ratios.

TABLE 13

| Samples | Tumor to kidney ratio |
|---|---|
| radiolabeled DARPin02 | 1:37 |
| radiolabeled DARPin02 + Cold-DARPin01 | 1:17 |
| radiolabeled DARPin02 + Cold-DARPin05 | 1:23 |
| radiolabeled DARPin02 + Cold-DARPin06 | 1:23 |
| radiolabeled DARPin02 + Cold-DARPin07 | 1:22 |
| radiolabeled DARPin02 + Cold-DARPin08 | 1:21 |
| radiolabeled DARPin02 + Cold-DARPin09 | 1:22 |
| radiolabeled DARPin02 + Cold-DARPin10 | 1:25 |
| radiolabeled DARPin02 + Cold-DARPin11 | 1:31 |

Statistical analyses were performed with Prism 9 (GraphPad Software LLC, San Diego, CA, USA).

### Example 4: Comparative Study

In this study, the protective effect on kidneys provided by the co-administration of cold blocker DARPin01 was compared to two compounds known to have kidney protective effects when administered in combination with radiopharmaceutical compounds, i.e an alpha-1-microglobulin variant (also herein referred to as comparative compound 1) and Gelofusine (comparative compound 2).

The HER2-specific DARPin04 was used as radiolabeled (hot) compound, conjugated to DTPA and subsequently loaded with ¹¹¹In as described in Example 1. The radiochemical purity was assessed by thin layer chromatography (mini-Gita dual Radio-TLC Imaging Scanner (Elysia-Raytest); Chromatographic Paper: iTLC-SG; Mobile phase: 0.1M Sodium Citrate Buffer pH 7-7.4). Radiolabeled DARPin04 was co-administered with Cold-DARPin01 (Group 1), with alpha-1-microglobulin variant (Group 2) or with Gelofusine (Group 3), as detailed in Table 14. Compounds were formulated in PBS+0.05% Tween-20.

Tumor cells were SKOV-3 cells, which present epithelial morphology and are derived from human ovarian adenocarcinoma. Cells were grown in McCoy's Medium supplemented with 10% FBS, 100 U/mL Penicillin and 100 μg/mL of Streptomycin. Female Crl:CD1-Foxn1nu mice (Charles River, Germany) were used, aged 7-8 weeks old at the day of cell inoculation. Once tumor volumes reach a volume of 200 to 500 mm³, animals were randomized in four different groups. Mice were anesthetized with 2-2.5% isoflurane to allow intravenous injection (iv) into the caudal vein via a catheter. For each group, injected activity of the radiolabeled DARPin04 was 10-15 MBq/mouse.

Gelofusine (Physiogel®, B. Braun Medical AG) was obtained commercially and the alpha-1-microglobulin variant of SEQ ID NO: 18 was recombinantly produced and purified following standard protein expression and purification protocols. In short, the alpha-1-microglobulin variant of SEQ ID NO: 18 was recombinantly expressed as inclusion bodies in E. coli strain BL21, with the alpha-1-microglobulin gene expressed under the control of a IPTG-inducible promoter. A pre-culture of the expression vector was inoculated into fresh TB/amp50 medium, cultivated at 37° C. under shaking and expression was started at an OD600 of 0.2 by addition of 1 mM IPTG. Cells were harvested after 4.5 h expression by centrifugation, resuspended in lysis buffer and stored at −20° C. Cells were then disrupted by sonication, the lysate was centrifuged at 18'000 g for 30 min, the supernatant was disposed and the protein was collected as insoluble pellet. The pellet was stored at −20° C. until further use. The insoluble pellets were then resuspended in GuHCL-IMAC buffer (6M Gu-HCl, 20 mM Tris; pH 8.0) for 1 h under light agitation, before 2×30 min centrifugation at 15'000 g. Imidazole was added to a final concentration of 15 mM and the supernatant was added to IMAC resins and rotated at room temperature for 1 h. The resin was separated from the supernatant, poured into an empty gravity-flow column and the resin was washed with ~10 resin volumes of GuHCI IMAC wash buffer (6M Gu-HCl, 20 mM Tris; pH 8.0, 15 mM imidazole). The protein was then eluted by addition of 1 resin volume of elution buffer (6M Gu-HCl, 20 mM Tris; pH 8.0, 500 mM imidazole). All eluates containing the desired protein were pooled, put on ice and mixed with an equal volume of freshly prepared 5 mM reduced glutathione. This reduced eluate was then dripped slowly in 10 volumes of ice-cold refolding buffer (500 mM L-Arg, 650 mM NaCl, 2 mM EDTA, 100 mM Tris, pH 8.0, 1 mM L-glutathion (oxidized)) while stirring. The solution was stirred for 1 h at 4° C. Then, the protein solution was re-buffered into TBS500 buffer (50 mM Tris-HCl, 500 mM NaCl, pH 8) using a tangential flow filtration device with a 5 kDa MWCO membrane, by first concentrating the diluted, refolded protein to ~100 mL, then exchanging the buffer via the passage of ~7 volumes of 1×TBS500. The rebuffered protein was then recovered from the TFF device and purified further over an immobilized metal affinity column (IMAC) followed by size exclusion chromatography (HiLoad 26/600 Superdex 200 column) using an Aekta Express system.

Radioactivity of tissue samples of interest was measured 4 h post injection with a γ-counter instrument (Wizard2 2470, Perkin Elmer) following mice sacrifice and tissue weighting.

TABLE 14

| | | | Mice | Dosing per |
|---|---|---|---|---|
| Group | Treatment | Construct details | number | mouse |
| 1 | radiolabeled | GS-DARPin04-GSGSC- | 6 | 20 μg |
| | DARPin04 | DTPA-¹¹¹In | | (1.5 nmol) |
| 2 | radiolabeled | GS-DARPin04-GSGSC- | 6 | 20 μg |
| | DARPin04 | DTPA-¹¹¹In | | (1.5 nmol) |
| | Cold-DARPin01 | MRGSHis6GS-DARPin01 | | 1000 μg |
| | | | | (69.2 nmol) |
| 3 | radiolabeled | GS-DARPin04-GSGSC- | 6 | 20 μg |
| | DARPin04 | DTPA-¹¹¹In | | (1.5 nmol) |
| | Comparative | alpha-1-microglobulin | | 1000 μg |
| | compound 1 | variant | | (45.2 nmol) |
| 4 | radiolabeled | GS-DARPin04-GSGSC- | 6 | 20 μg |
| | DARPin04 | DTPA-¹¹¹In | | (1.5 nmol) |
| | Comparative | Gelofusine | | 3200 μg |
| | compound 2 | | | (160 |
| | | | | mg/kg) |

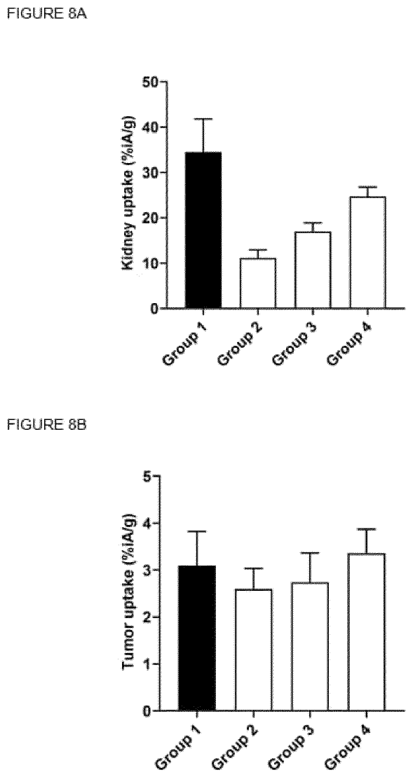

The measured radioactivity is shown in FIG. 8, where FIG. 8A shows the mean accumulation in the kidneys and FIG. 8B the mean accumulation in the tumors. Compared to the comparative compounds 1 and 2, co-administration of Cold-DARPin01 induces the highest reduction in kidney accumulation. All tested blockers show a negligible impact on the tumor uptake. The percentage of reduction for each Group 2 to 4 in comparison to the non-blocked Group 1 is shown in Table 15, values are computed as in section 1.5 of Example 1.

TABLE 15

| | | Reduction in kidney |
|---|---|---|
| | | uptake blocked |
| Blocked | non-blocked | vs. non-blocked |
| radiolabeled DARPin04 + | radiolabeled DARPin04 | 67.5% |
| Cold-DARPin01 | | |
| radiolabeled DARPin04 + | radiolabeled DARPin04 | 50.7% |
| Comparative compound 1 | | |
| radiolabeled DARPin04 + | radiolabeled DARPin04 | 28.4% |
| Comparative compound 2 | | |

The corresponding average tumor to kidney ratios are shown in Table 16.

TABLE 16

| | Tumor to |
|---|---|
| Samples | kidney ratio |
| radiolabeled DARPin04 | 1:11 |
| radiolabeled DARPin04 + Cold-DARPin01 | 1:4 |
| radiolabeled DARPin04 + Comparative compound 1 | 1:6 |
| radiolabeled DARPin04 + Comparative compound 2 | 1:7 |

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and GenBank sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize or be able to ascertain using no more than routine experimentation, many equivalents to the specific embodiments of the invention described herein. Such equivalents are intended to be encompassed by the following claims.

SEQUENCES

| SEQ | | |
|---|---|---|
| ID NO | Description | Sequence |
| 1 | Designed ankyrin | DLGKKLLEAARAGQDDEVRELLKA |
| | repeat domain | GADVNAKDKDGYTPLHLAAREGHL |
| | | EIVEVLLKAGADVNAKDKDGYTPL |
| | | HLAAREGHLEIVEVLLKAGADVNA |
| | | QDKSGKTPADLAADAGHEDIAEVL |
| | | QKAA |
| 2 | Designed ankyrin | DLGKKLLEAARAGQDDEVRELLKA |
| | repeat domain | GADVNAKDEYGLTPLYLATAHGHL |
| | | EIVEVLLKAGADVNAVDAIGFTPL |
| | | HLAAFIGHLEIAEVLLKAGADVNA |
| | | QDKFGKTPADIAAGAGNEDIAEVL |
| | | QKAA |
| 3 | Designed ankyrin | DLGIKLLFAAAKGQDDEVRELLKA |
| | repeat domain | GADVNAKDFQGVTPLHIAAQSGHL |
| | | EIVEVLLKAGADVNAKDVTGDTPL |
| | | HLAAQHGHLEIVEVLLKAGADVNA |
| | | QDERGWTPADLAADWGHEDIAEVL |
| | | QKAA |
| 4 | Designed ankyrin | DLGTQLLRAAESGQLDEVRELLKA |
| | repeat domain | GADVNAQDEYGLTPLYLATAHGHL |
| | | EIVTVLLEAGADVNAQDAIGFTPL |
| | | HLAAFIGHLEIATVLLEAGADVNA |
| | | QDKFGKTPADIAAGAGNEDIAEVL |
| | | QKAA |
| 5 | N-terminal | DLGKKLLEAARAGQDDEVRELLKA |
| | capping module | GADVNA |
| 6 | C-terminal | QDKSGKTPADLAADAGHEDIAEVL |
| | capping module | QKAA |
| 7 | Ankyrin repeat | KDKDGYTPLHLAAREGHLEIVEVL |
| | module | LKAGADVNA |
| 8 | Tag | MRGSHHHHHHENLYFQ |
| 9 | Tag | MRGSHHHHHHGS |
| 10 | Tag | GSGSC |
| 11 | Designed ankyrin | DLGKKLLRAARAGQDDEVRELLKA |
| | repeat domain | GADVNAKDKDGYTPLHLAAREGHL |
| | | EIVEVLLKAGADVNAKDKDGYTPL |
| | | HLAAREGHLEIVEVLLKAGADVNA |
| | | QDKSGKTPADLAARAGHEDIAEVL |
| | | QKAA |
| 12 | Designed ankyrin | DLGKKLLRAARKGQDDEVRELLKA |
| | repeat domain | GADVNAKDKDGYTPLHKAAREGHL |
| | | EIVEVLLKAGADVNAKDKDGYTPL |
| | | HKAAREGHLEIVEVLLKAGADVNA |
| | | QDKSGKTPADLAARKGHEDIAEVL |
| | | QKAA |
| 13 | Designed ankyrin | DLGKKLLRAARAGQLDTVRELLKA |
| | repeat domain | GADVNAKDKDGYTPLHKAARSGHL |
| | | EIVEVLLKAGADVNAKDKDGYTPL |
| | | HKAARSGHLEIVEVLLKAGADVNA |
| | | QDKSGKTPADLAARAGHEDIAEVL |
| | | QKAA |
| 14 | Designed ankyrin | DLGKKLLQAARAGQLDEVRELLKA |
| | repeat domain | GADVNAKDKSGYTPLHKAAERGHL |
| | | EIVEVLLKAGADVNAKDKSGYTPL |
| | | HKAAERGHLEIVEVLLKAGADVNA |
| | | QDKSGKTPADLAADRGHKQIAEVL |
| | | QKAA |
| 15 | Designed ankyrin | DLGKKLLEAARAGQDDKVRELLKA |
| | repeat domain | GAKVNAKDKDGYTPLHLAAREGHR |
| | | KIVEVLLKAGADVNAKDKDGYTPL |
| | | HLAAREGHRKIVEVLLKAGADVNA |
| | | QDKSGKTPADLAADAGHEKIAKVL |
| | | QKAA |
| 16 | Designed ankyrin | DLGKKLLRAARAGQDDEVRELLKA |
| | repeat domain | GADVNAKDKDGKTPLHKAARYGHL |
| | | EIVEVLLAKGADVNAKDKDGKTPL |
| | | HKAARYGHLEIVEVLLAKGADVNA |
| | | QDKSGKTPADLAARAGHEDIAEVL |
| | | QKAA |
| 17 | Designed ankyrin | DLGKKLLRAARAGQDDEVRELLKA |
| | repeat domain | GADVNAKDKDGKTPLHKAARYGHL |
| | | EIVEVLLAKGADVNAKDKDGKTPL |
| | | HKAARYGHLEIVEVLLAKGADVNA |
| | | QDKSGKTPADLAARAGHEDIAKVL |
| | | QKAA |
| 18 | Comparative | MHHHHHHDDDDKGPVPTPPDNIQV |
| | compound 1 | QENFDISRIYGKWYNLAIGSTCPW |
| | | LKKIMDRMTVSTLVLGEGATEAEI |
| | | SMTSTHWRKGVCEETSGAYEKTDT |
| | | DGKFLYHKSKWDITMESYVVHTNY |
| | | DEYAIFLTKKFSRHHGPTITAKLY |
| | | GRAPQLRETLLQDFRVVAQGVGIP |
| | | EDSIFTMADRGECVPGEQEPEPIL |
| | | IPR |

1. A designed ankyrin repeat domain for use in reducing accumulation of a therapeutic and/or diagnostic agent in the kidney of a subject treated with said agent, wherein the designed ankyrin repeat domain is administered to the subject in an amount effective to reduce accumulation of said therapeutic and/or diagnostic agent in the kidney.

2. The designed ankyrin repeat domain for use according to claim 1, wherein said subject is treated with said agent by systemic administration of said agent.

3. The designed ankyrin repeat domain for use according to claim 1, wherein the reduction of accumulation of said agent in the kidney is measured between about 1 hour and about 24 hours after treatment of the subject with the agent.

4. The designed ankyrin repeat domain for use according to claim 1, wherein the designed ankyrin repeat domain is administered concomitantly with the agent.

5. The designed ankyrin repeat domain for use according to claim 1, wherein the designed ankyrin repeat domain is administered to the subject at a molar ratio of designed ankyrin repeat domain to agent of about 1:1 or higher, about 5:1 or higher, about 20:1 or higher, about 50:1 or higher, about 100:1 or higher, about 500:1 or higher, about 1000:1 or higher, about 1500:1 or higher, about 2000:1 or higher, about 2500:1 or higher, about 3000:1 or higher, or about 3500:1 or higher, about 4000:1 or higher, about 4500:1 or higher, about 5000:1 or higher, about 5500:1 or higher, or about 6000:1 or higher.

6. The designed ankyrin repeat domain for use according to claim 1, wherein the accumulation of said agent in the kidney is reduced by at least 10%, at least 20%, at least 30%, at least 40% or at least 50% as compared to the accumulation of said agent in the kidney of a subject treated with said agent as a control without administration of the designed ankyrin repeat domain.

7. The designed ankyrin repeat domain for use according to claim 1, wherein the designed ankyrin repeat domain does not specifically bind to a target with a dissociation constant (K_{D}) of 10⁻⁷M or below.

8. The designed ankyrin repeat domain for use according to claim 1, wherein the designed ankyrin repeat domain comprises:
(a) an N-terminal capping module having the amino acid sequence of SEQ ID NO: 5 or any variant thereof having at least 60%, at least 70%, at least 80% or at least 90% sequence identity with SEQ ID NO: 5, and/or
(b) a C-terminal capping module having the amino acid sequence of SEQ ID NO: 6 or any variant thereof having at least 60%, at least 70%, at least 80% or at least 90% sequence identity with SEQ ID NO: 6, and/or
(c) one or more internal repeat module(s) each independently having the amino acid sequence of SEQ ID NO: 7 or any variant thereof having at least 60%, at least 70%, at least 80% or at least 90% sequence identity with SEQ ID NO: 7.

9. The designed ankyrin repeat domain for use according to claim 1, wherein the designed ankyrin repeat domain comprises:
(a) an N-terminal capping module having the amino acid sequence of SEQ ID NO: 5 or any variant of SEQ ID NO: 5 wherein:
(i) up to 4 amino acids selected from the randomized positions corresponding to positions 4, 8, 11 and 12 of SEQ ID NO: 5 are substituted by a different amino acid, and
(ii) up to 15 amino acids selected from the non-randomized positions corresponding to positions 1, 2, 3, 5, 6, 7, 9, 10, 13, 14, 15, 16, 17, 18, 19, 20, 21, 22, 23, 24, 25, 26, 27, 28, 29 and 30 of SEQ ID NO: 5 are substituted by a different amino acid, and/or
(b) a C-terminal capping module having the amino acid sequence of SEQ ID NO: 6 or any variant of SEQ ID NO: 6 wherein:
(i) up to 5 amino acids selected from the randomized positions corresponding to positions 3, 4, 6, 14 and 15 of SEQ ID NO: 6 are substituted by a different amino acid, and
(ii) up to 15 amino acids selected from the non-randomized positions corresponding to positions 1, 2, 5, 7, 8, 9, 10, 11, 12, 13, 16, 17, 18, 19, 20, 21, 22, 23, 24, 25, 26, 27 and 28 of SEQ ID NO: 6 are substituted by a different amino acid, and/or
(c) one or more internal repeat module(s) each independently having the amino acid sequence of SEQ ID NO: 7 or any variant of SEQ ID NO: 7 wherein:
(i) up to 6 amino acids selected from the randomized positions corresponding to positions 3, 4, 6, 11, 14 and 15 of SEQ ID NO: 7 are substituted by a different amino acid, and
(ii) up to 15 amino acids selected from the non-randomized positions corresponding to positions 1, 2, 5, 7, 8, 9, 10, 12, 13, 16, 17, 18, 19, 20, 21, 22, 23, 24, 25, 26, 27, 28, 29, 30, 31, 32 and 33 of SEQ ID NO: 7 are substituted by a different amino acid.

10. The designed ankyrin repeat domain for use according to claim 1, wherein the designed ankyrin repeat domain comprises two or more internal repeat modules, preferably two internal repeat modules.

11. The designed ankyrin repeat domain for use according to claim 10, wherein the internal repeat modules comprised in the designed ankyrin repeat domain have at least 80%, at least 85%, at least 90%, at least 91%, at least 92%, at least 93%, at least 94%, at least 95%, at least 96%, at least 97%, at least 98%, at least 99% or 100% sequence identity between each other.

12. The designed ankyrin repeat domain for use according to claim 1, wherein the designed ankyrin repeat domain comprises an amino acid sequence selected from the group consisting of (1) SEQ ID NO: 1 and (2) sequences with at least 80% amino acid sequence identity with SEQ ID NO: 1.

13. The designed ankyrin repeat domain for use according to claim 1, wherein the therapeutic and/or diagnostic agent comprises a binding moiety and a drug moiety.

14. The designed ankyrin repeat domain for use according to claim 13, wherein said drug moiety is a toxin.

15. The designed ankyrin repeat domain for use according to claim 14, wherein said toxin is a radionuclide.

16. The designed ankyrin repeat domain for use according to claim 14, wherein said toxin is a cytotoxin.

17. The designed ankyrin repeat domain for use according to claim 13, wherein said binding moiety comprises a designed ankyrin repeat domain with binding specificity for a target.

18. A recombinant protein for use in reducing accumulation of a therapeutic and/or diagnostic agent in the kidney of a subject treated with said agent, wherein the recombinant protein is administered to the subject in an amount effective to reduce accumulation of said therapeutic and/or diagnostic agent in the kidney, and wherein said recombinant protein comprises the designed ankyrin repeat domain for use according to claim 1.

19. The recombinant protein for use according to claim 18, wherein the molecular weight of the recombinant protein is 69 kDa or less, 65 kDa or less, 60 kDa or less, 55 kDa or less, 50 kDa or less, 45 kDa or less, 40 kDa or less, 35 kDa or less, or 30 kDa or less.

20. An isolated nucleic acid encoding the designed ankyrin repeat domain as defined in claim 1 or the recombinant protein as defined in claim 18.

21. A recombinant expression vector comprising the nucleic acid according to claim 20.

22. A host cell comprising the recombinant expression vector according to claim 21.

23. A pharmaceutical composition comprising one or more of: (i) the designed ankyrin repeat domain as defined in claim 1, (ii) the recombinant protein as defined in claim 18, (iii) the nucleic acid according to claim 20, and/or (iv) the recombinant expression vector according to claim 21, and optionally at least one pharmaceutically acceptable carrier or diluent.

24. A method of reducing accumulation of a therapeutic and/or diagnostic agent in the kidney of a subject treated with said agent, the method comprising the step of administering to the subject an effective amount of a designed ankyrin repeat domain.

25. The method according to claim 24, wherein the designed ankyrin repeat domain does not specifically bind to a target with a dissociation constant (K_{D}) of 10⁻⁷M or below.

26. A recombinant protein comprising a designed ankyrin repeat domain, wherein the designed ankyrin repeat domain does not specifically bind to a target with a dissociation constant (K_{D}) of 10⁻⁷M or below, for use as a medicament.


SEQUENCE LISTING (18 sequences)

SEQ ID NO: 1 - protein, 124 aa, synthetic construct
DLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAA

SEQ ID NO: 2 - protein, 124 aa, synthetic construct
DLGKKLLEAARAGQDDEVRELLKAGADVNAKDEYGLTPLYLATAHGHLEIVEVLLKAGADVNAVDAIGFTPLHLAAFIGHLEIAEVLLKAGADVNAQDKFGKTPADIAAGAGNEDIAEVLQKAA

SEQ ID NO: 3 - protein, 124 aa, synthetic construct
DLGIKLLFAAAKGQDDEVRELLKAGADVNAKDFQGVTPLHIAAQSGHLEIVEVLLKAGADVNAKDVTGDTPLHLAAQHGHLEIVEVLLKAGADVNAQDERGWTPADLAADWGHEDIAEVLQKAA

SEQ ID NO: 4 - protein, 124 aa, synthetic construct
DLGTQLLRAAESGQLDEVRELLKAGADVNAQDEYGLTPLYLATAHGHLEIVTVLLEAGADVNAQDAIGFTPLHLAAFIGHLEIATVLLEAGADVNAQDKFGKTPADIAAGAGNEDIAEVLQKAA

SEQ ID NO: 5 - protein, 30 aa, synthetic construct
DLGKKLLEAARAGQDDEVRELLKAGADVNA

SEQ ID NO: 6 - protein, 28 aa, synthetic construct
QDKSGKTPADLAADAGHEDIAEVLQKAA

SEQ ID NO: 7 - protein, 33 aa, synthetic construct
KDKDGYTPLHLAAREGHLEIVEVLLKAGADVNA

SEQ ID NO: 8 - protein, 16 aa, synthetic construct
MRGSHHHHHHENLYFQ

SEQ ID NO: 9 - protein, 12 aa, synthetic construct
MRGSHHHHHHGS

SEQ ID NO: 10 - protein, 5 aa, synthetic construct
GSGSC

SEQ ID NO: 11 - protein, 124 aa, synthetic construct
DLGKKLLRAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAARAGHEDIAEVLQKAA

SEQ ID NO: 12 - protein, 124 aa, synthetic construct
DLGKKLLRAARKGQDDEVRELLKAGADVNAKDKDGYTPLHKAAREGHLEIVEVLLKAGADVNAKDKDGYTPLHKAAREGHLEIVEVLLKAGADVNAQDKSGKTPADLAARKGHEDIAEVLQKAA

SEQ ID NO: 13 - protein, 124 aa, synthetic construct
DLGKKLLRAARAGQLDTVRELLKAGADVNAKDKDGYTPLHKAARSGHLEIVEVLLKAGADVNAKDKDGYTPLHKAARSGHLEIVEVLLKAGADVNAQDKSGKTPADLAARAGHEDIAEVLQKAA

SEQ ID NO: 14 - protein, 124 aa, synthetic construct
DLGKKLLQAARAGQLDEVRELLKAGADVNAKDKSGYTPLHKAAERGHLEIVEVLLKAGADVNAKDKSGYTPLHKAAERGHLEIVEVLLKAGADVNAQDKSGKTPADLAADRGHKQIAEVLQKAA

SEQ ID NO: 15 - protein, 124 aa, synthetic construct
DLGKKLLEAARAGQDDKVRELLKAGAKVNAKDKDGYTPLHLAAREGHRKIVEVLLKAGADVNAKDKDGYTPLHLAAREGHRKIVEVLLKAGADVNAQDKSGKTPADLAADAGHEKIAKVLQKAA

SEQ ID NO: 16 - protein, 124 aa, synthetic construct
DLGKKLLRAARAGQDDEVRELLKAGADVNAKDKDGKTPLHKAARYGHLEIVEVLLAKGADVNAKDKDGKTPLHKAARYGHLEIVEVLLAKGADVNAQDKSGKTPADLAARAGHEDIAEVLQKAA

SEQ ID NO: 17 - protein, 124 aa, synthetic construct
DLGKKLLRAARAGQDDEVRELLKAGADVNAKDKDGKTPLHKAARYGHLEIVEVLLAKGADVNAKDKDGKTPLHKAARYGHLEIVEVLLAKGADVNAQDKSGKTPADLAARAGHEDIAKVLQKAA

SEQ ID NO: 18 - protein, 195 aa, synthetic construct
MHHHHHHDDDDKGPVPTPPDNIQVQENFDISRIYGKWYNLAIGSTCPWLKKIMDRMTVSTLVLGEGATEAEISMTSTHWRKGVCEETSGAYEKTDTDGKFLYHKSKWDITMESYVVHTNYDEYAIFLTKKFSRHHGPTITAKLYGRAPQLRETLLQDFRVVAQGVGIPEDSIFTMADRGECVPGEQEPEPILIPR